(19) United States
(12) Patent Application Publication
(10) Pub. No.: US 2025/0262261 A1
(43) Pub. Date: Aug. 21, 2025

(54) COMPOSITIONS AND METHODS FOR TREATING NEUROLOGICAL DISORDERS

(71) Applicant: Neurotech International Ltd, Nedlands (AU)

(72) Inventors: Allan William Cripps, Queensland (AU); Esra Isikgel, Victoria (AU); Alexandra Elizabeth Marion Andrews, Nedlands (AU); Thomas George Duthy, Beaumont (AU)

(73) Assignee: Neurotech International Ltd, Nedlands (AU)

(21) Appl. No.: 18/698,605

(22) PCT Filed: Oct. 11, 2022
(86) PCT No.: PCT/AU2022/051220

§ 371 Date: Apr. 4, 2024

(30) Foreign Application Priority Data
Oct. 11, 2021 (AU) 2021903256
Mar. 16, 2022 (AU) 2022900640
Jun. 22, 2022 (AU) 2022901710

(51) Int. Cl. A61K 36/185; A61K 31/00; A61K 47/44; A61P 25/28

(52) U.S. Cl. CPC A61K 36/3482; A61K 31/658; A61K 47/44; A61P 25/28; A61K 2236/15; A61K 2236/333; A61K 2236/35; A61K 2236/53

(57) ABSTRACT

The present invention relates to compositions comprising cannabinoids. The present invention also relates to pharmaceutical compositions, dosage forms and methods of treating neurological disorders by administering the composition to a patient in need thereof.

### RELATED APPLICATIONS

This application is a national phase filing under 35 U.S.C. 371 of International Application No. PCT/AU2022/051220, filed Oct. 11, 2022, which claims the benefit of and priority to AU Application No. 2021903256, filed on Oct. 11, 2021, AU Application No. 2022900640, filed Mar. 16, 2022, and AU Application No. 2022901710, filed Jun. 22, 2022, the contents of each of which are incorporated herein by reference in their entireties.

### FIELD OF THE INVENTION

The present invention relates to compositions comprising cannabinoids. The present invention also relates to pharmaceutical compositions, dosage forms and methods of treating neurological disorders by administering the composition to a patient in need thereof.

### BACKGROUND

The following discussion of the background art is intended to facilitate an understanding of the present invention only. The discussion is not an acknowledgement or admission that any of the material referred to is or was part of the common general knowledge as at the priority date of the application.

### A. Neuroinflammation

Neuroinflammation refers to the process whereby the brain's innate immune system is triggered following an inflammatory challenge such as those posed by injury, infection, exposure to a toxin, neurodegenerative disease, or aging. Neuroinflammation is implicated in contributing to a variety of neurologic and somatic illnesses including Alzheimer's disease (AD), Parkinson's disease (PD), multiple sclerosis, amyotrophic lateral sclerosis, cerebral ischemia, traumatic brain injury, rheumatoid arthritis, chronic migraine, epilepsy, autism spectrum disorder (ASD), attention deficit hyperactivity disorder (ADHD), cerebral palsy and relevant subtypes, neuropathic pain, and depression.

In the central nervous system (CNS), the innate immune response plays a significant role in both physiological and pathological conditions. CNS diseases including traumatic brain injury, ischemic stroke, brain tumor, and cerebrovascular and neurodegenerative diseases trigger a cascade of events broadly defined as neuroinflammation, which is characterized by the activation of the microglia and astrocyte population. On the other hand, microglial and astrocyte activation, T lymphocyte infiltration, and overproduction of inflammatory cytokines have been demonstrated in association with neuronal alteration in both animal and human tissues. Neuroinflammation is therefore an important topic in contemporary neuroscience.

Inflammatory cytokines/markers or proinflammatory cytokines/markers are types of signaling molecules that are secreted from immune cells like helper T cells and macrophages and certain other cell types that promote the process of neuro-inflammation and general inflammatory processes. These include interleukin-1 (IL-1), IL-12, and IL-18, tumor necrosis factor alpha (TNF-α), interferon gamma (IFNγ) and granulocyte-macrophage colony stimulating factor (GM-CSF). These inflammatory cytokines are predominantly produced by and involved in the upregulation of inflammatory reactions and play an important role in mediating the innate immune response.

### B. Neurological Disorders

Examples of neurological disorders that are “neuro-inflammatory based” include: Alzheimer's disease (Alzheimer's disease is the most prevalent chronic, progressive neurodegenerative disease, and cause of dementia); Parkinson's disease; multiple sclerosis; amyotrophic lateral sclerosis; cerebral ischemia; traumatic brain injury; rheumatoid arthritis; chronic migraine; epilepsy; autism spectrum disorder; attention deficit hyperactivity disorder; cerebral palsy and relevant subtypes; neuropathic pain; and depression.

### C. Microglia Activation|Neurodegeneration

Microglia cells are the unique residential macrophages of the central nervous system (CNS). They play an important role during CNS development and adult homeostasis. They have a major contribution to adult neurogenesis and neuroinflammation (Zhan Y., Paolicelli R. C., Sforazzini F., et al. Deficient neuron-microglia signaling results in impaired functional brain connectivity and social behavior. Nature Neuroscience. 2014; 17(3):400-406; Guruswamy R, ElAli A. Complex Roles of Microglial Cells in Ischemic Stroke Pathobiology: New Insights and Future Directions. Int J Mol Sci. 2017; 18:18). Thus, they participate in the pathogenesis of neurodegenerative diseases and contribute to aging. They play an important role in sustaining and breaking the blood-brain barrier. As innate immune cells, they contribute substantially to the immune response against infectious agents affecting the CNS (Xiong X Y, Liu L, Yang Q W. Functions and mechanisms of microglia/macrophages in neuroinflammation and neurogenesis after stroke. Prog Neurobiol. 2016; 142:23-44). They also play a major role in the growth of tumours of the CNS. Microglia are consequently the key cell population linking the nervous and the immune system (Xiong X Y, Liu L, Yang Q W. Functions and mechanisms of microglia/macrophages in neuroinflammation and neurogenesis after stroke. Prog Neurobiol. 2016; 142:23-44).

Under physiological conditions, ramified, resting microglia provides a neuroprotective environment (David S, Greenhalgh A D, Kroner A. Macrophage and microglial plasticity in the injured spinal cord. Neuroscience. 2015; 307:311-18; Bieber K, Autenrieth S E. Insights how monocytes and dendritic cells contribute and regulate immune defense against microbial pathogens. Immunobiology. 2015; 220:215-26). However, most CNS pathologies, as well as regenerative efforts, include activation of microglia with corresponding inflammatory events (Hoogland I C, Houbolt C, van Westerloo D J, van Gool W A, van de Beek D. Systemic inflammation and microglial activation: systematic review of animal experiments. J Neuroinflammation. 2015; 12:114; Ascoli B M, Géa L P, Colombo R, Barbé-Tuana F M, Kapczinski F, Rosa A R. The role of macrophage polarization on bipolar disorder: identifying new therapeutic targets. Aust N Z J Psychiatry. 2016; 50:618-30; Cherry J D, Olschowka J A, O'Banion M K. Neuroinflammation and M2 microglia: the good, the bad, and the inflamed. J Neuroinflammation. 2014; 11:98). Activated, inflammatory microglia are thus neurotoxic and kill neurons by engulfing them or releasing various neurotoxic molecules and factors, including reactive oxygen species (ROS), glutamate, Fas-ligand, tumour necrosis factor α (TNFα) and others (Loane D J, Kumar A. Microglia in the TBI brain: the good, the bad, and the dysregulated. Exp Nurol. 2016; 275:316-27; Nakagawa Y, Chiba K. Diversity and plasticity of microglial cells in psychiatric and neurological disorders. Pharmacol Ther. 2015; 154:21-35).

Activated microglia driving chronic neuroinflammation have also been shown to substantially contribute to aging of the CNS (Loane D J, Kumar A. Microglia in the TBI brain: the good, the bad, and the dysregulated. Exp Neurol. 2016; 275:316-27), chronic neuropathic pain (Orihuela R, McPherson C A, Harry G J. Microglial M1/M2 polarization and metabolic states. Br J Pharmacol. 2016; 173:649-65) and mental diseases (Orihuela R, McPherson C A, Harry G J. Microglial M1/M2 polarization and metabolic states. Br J Pharmacol. 2016; 173:649-65) and neurodegenerative diseases, including Alzheimer's disease (Nakagawa Y, Chiba K. Diversity and plasticity of microglial cells in psychiatric and neurological disorders. Pharmacol Ther. 2015; 154:21-35) Parkinson's disease (Orihuela R, McPherson C A, Harry G J. Microglial M1/M2 polarization and metabolic states. Br J Pharmacol. 2016; 173:649-65) amyotrophic lateral sclerosis (ALS) and multiple sclerosis. Aging goes in parallel with systemic chronic activation of the immune system and polarization towards a low-level inflammatory status (Ransohoff R M. A polarizing question: do M1 and M2 microglia exist?Nat Neurosci. 2016; 19:987-91; Tang Y, Le W. Differential Roles of M1 and M2 Microglia in Neurodegenerative Diseases. Mol Neurobiol. 2016; 53:1181-94).

### D. Medicinal Applications of Cannabis

Cannabis sativa L. has a tradition of medical use. Medicinal cannabis has attracted significant interest due to its anti-inflammatory, anti-oxidative and anti-necrotic protective effects, as well as displaying a favourable safety and tolerability profile in humans, making it a promising candidate in many therapeutic avenues. However, clinical use has been restricted because of untoward effects on the central nervous system and the possibility of abuse and addiction. The plant exudes a resin containing a mix of cannabinoids with two principal components, Δ9-tetrahydrocannabinol (THC) and cannabidiol (CBD). The structure of CBD was described in the 1960s and has garnered attention due to the lack of psychotropic activity. Because of its excellent tolerability in humans, the lack of psychoactive action, and low abuse potential, it seems ideal for clinical trial.

### E. Cannabinoids

In addition to its good safety profile and the lack of psychoactive effects, CBD also presents a wide range of therapeutic effects. Several experimental in vitro and in vivo studies demonstrate anti-inflammation and immune modifying, anti-psychotic, analgesic and anti-epileptic actions. For these reasons, CBD is currently one of the most studied cannabinoids. Compared to Δ9-THC, CBD shows a low affinity for cannabinoid receptor type 1 (CB₁) and type 2 (CB₂). CB₁ receptors are mainly found in the terminals of central and peripheral neurons, and CB₂ receptors primarily in immune cells. Several in vitro studies have shown that CBD, at low concentrations, has weak CB₁ and CB₂ antagonistic effect.

Studies suggest that CBD behaves as a negative allosteric modulator of CB₁, meaning that CBD does not activate the receptor directly but alters the potency and efficacy of CBD1's orthosteric ligands: Δ9-THC and 2-arachidonoylglycerol (2-AG). These preliminary results need further validation but may explain the ability of CBD to antagonise some of the effects of Δ9-THC reported in vitro, in vivo and human clinical studies. It has also been suggested that the role of CBD as an allosteric modulator of CB₁ can explain its therapeutic role in the treatment of central and peripheral nervous system disorders. CBD has also shown to inhibit neutrophil chemotaxis and proliferation. It may also induce arachidonic acid release and reduce prostaglandin E2 (PGE2) and nitric oxide (NO) production.

However, not all physiological effects of CBD are mediated by cannabinoid receptors. CBD has numerous targets outside the endocannabinoid system, and its action independent of the cannabinoid receptor is the subject of recent pharmacological studies. Some effects, such as anti-inflammatory and immunosuppressive action, are mediated by more than one target. The anti-inflammatory, immunosuppressive effects are possibly mediated by activation of adenosine receptors, A_{1A} and A_{2A} and strychnine-sensitive α1 and α1β glycine receptors and the inhibition of the equilibrative nucleoside transporter. Furthermore, the activity of CBD may elicit different physiological effects from the same target. For example, the same glycine receptor is implicated in both anti-inflammation and suppression of neuropathic pain. While effects on serotonin 5HT1A receptors may generate anxiolytic, panicolytic and antidepressant effects, research has showed an in-depth review of the molecular pharmacology of CBD. Despite advances in the molecular pharmacology of CBD, the many pharmacological mechanisms of CBD remain uncharacterized.

Published studies in animals demonstrated that the oral bioavailability of cannabidiol has been shown to be approximately between 13-19%. Plasma and brain concentrations are dose-dependent in animals, and bioavailability is increased with various oil formulations. Cannabinoids undergo extensive first pass metabolism and its metabolites are mostly excreted via the kidneys.

Cannabinoids are metabolized extensively by the liver, where it is hydroxylated to 7-OH-CBD by P450 enzymes, predominantly by the CYP3A (2/4) and CYP2C (8/9/19) families of isozymes. This metabolite then undergoes significant further metabolism in the liver, and the resulting metabolites are excreted in the faeces and, to a much lesser extent, in the urine.

It is known that cannabidiol acts on cannabinoid (CB) receptors (CB1 and CB2) of the endocannabinoid system, which are found in numerous areas of the body, including the peripheral and central nervous systems, including the brain. The endocannabinoid system regulates many physiological responses of the body including pain, memory, appetite, and mood. More specifically, CB1 receptors can be found within the pain pathways of the brain and spinal cord where they may affect cannabidiol-induced analgesia and anxiolysis, and CB2 receptors have an effect on immune cells, where they may affect cannabidiol-induced anti-inflammatory processes.

Cannabidiol has been shown to act as a negative allosteric modulator of the cannabinoid CB1 receptor, the most abundant G-Protein Coupled Receptor (GPCR) in the body. Allosteric regulation of a receptor is achieved through the modulation of the activity of a receptor on a functionally distinct site from the agonist or antagonist binding site. The negative allosteric modulatory effects of cannabidiol are therapeutically important as direct agonists are limited by their psychomimetic effects while direct antagonists are limited by their depressant effects.

There has been some developments in the regulatory approval of CBD. Epidiolex® is a plant-derived, pharmaceutical grade cannabidiol (CBD) medication which attained FDA approval for use in the United States in 2018. Epidiolex® contains 100 mg of cannabidiol per milliliter (mL) of solutions and is taken orally twice daily. The Australian Therapeutic Goods Administration (TGA) approved Epidiolex in September 2020 for the treatment of seizures associated with Lennox-Gastaut syndrome (LGS) or Dravet syndrome in patients two years of age or older

There is a need in the art for improved cannabinoid compositions and effective treatments of neurological disorders. It is an objective of the invention to overcome one or more problems foreshadowed by the prior art.

### SUMMARY OF THE INVENTION

In a first aspect, the invention broadly resides in a composition comprising the following cannabinoids: about 50 w/w % of CBDA; and wherein all other cannabinoids come to about 15 w/w %.

In a preferred embodiment, the composition comprises the following cannabinoids:
w/w %
CBDA 40-60%;
CBD 1-5%;
CBG 1-10%;
CBDP 1-5%;
CBDB 1-5%;
CBGA 1-10%;
CBN 1-3%; and
THC<1%.

In another preferred embodiment, the composition comprises the following cannabinoids:
w/w %
CBDA 50%;
CBD 2%;
CBG 5%;
CBDP 2%;
CBDB 2%;
CBGA 5%;
CBN 1-3%; and
THC<0.3%.

In another preferred embodiment, the composition comprises the following cannabinoids:
w/w %
CBDA 49%;
CBD 2%;
CBG 5%;
CBDP 2%;
CBDB 2%;
CBGA 5%;
CBN 3%; and
THC<0.3%.

In another preferred embodiment, the composition comprises the following cannabinoids:
w/w %
CBDA 45%;
CBD 1%;
CBG 4%;
CBDP 1%;
CBDB 2%;
CBGA 4%;
CBN 2%; and
THC<0.2%.

In another preferred embodiment, the composition comprises the following cannabinoids:
w/w %
CBDA 45%;
CBD 1%;
CBG 4%;
CBDP 1%;
CBDB 2%;
CBGA 4%;
CBN 1%; and
THC<0.2%.

In a preferred embodiment, the composition comprises cannabinoids in amounts selected from the group consisting of any one of the above-mentioned embodiments.

In a second aspect, the invention is a pharmaceutical composition comprising the composition of the first aspect of the invention together with a pharmaceutically acceptable carrier.

In a third aspect, the invention is a dosage form comprising the composition of the first aspect of the invention.

In a fourth aspect, the invention is a method of treating a disorder, said method comprising administering to a patient in need thereof a therapeutically effective amount of the dosage form of the invention.

In a fifth aspect, the invention is the use of the composition of the invention in the manufacture of a medicament for the treatment of a disorder.

In a sixth aspect, the invention is a process of extracting the composition of the invention from cannabis plant material, said process comprising the steps of:
1) Grinding the cannabis plant material to a sufficient grind size;
2) Contacting the grind produced by step a) with oil;
3) Mixing the grind and oil for a sufficient time period to form a mixture;
4) Pressing the mixture to reclaim the oil;
5) Centrifuging the oil to further refine the oil; and
6) Collecting the oil extract in a suitable container/steel vessel.

In a seventh aspect, the invention is a process of extracting the composition of the invention from cannabis plant material, said process comprising the steps of:
1) Grinding the cannabis plant material to a sufficient grind size;
2) Contacting the grind produced by step a) with an alcohol;
3) Mixing the grind and alcohol for a sufficient time period to form a mixture;
4) Sonicating the mixture;
5) Centrifuging the mixture; and
6) Collecting the alcohol extract in a suitable container/steel vessel.

In an eighth aspect, the invention is the product produced from the process of the invention

In a ninth aspect, the invention is a kit comprising the dosage form of the invention together with instructions for its use.

In a ninth aspect, the invention includes a composition, method and process as described by the examples following.

Further features of the present invention are more fully described in the following description of several non-limiting embodiments thereof. This description is included solely for the purposes of exemplifying the present invention. It should not be understood as a restriction on the broad summary, disclosure or description of the invention as set out above.

### BRIEF DESCRIPTION OF THE DRAWINGS

Below is a brief description of each of the figures and drawings.

FIG. 1 is a UPLC mass spectrometry chromatogram of the cannabinoid standard mixture (10 ppm each) in; a) positive; and b) negative ionization mode.

FIG. 2 is an in-source fragmentation of CBD and CBG from the reference solution.

FIG. 3 shows mass spectrometry chromatograms for NT1164.

FIG. 4 shows quad mass spectrometry chromatograms of CBD variants of NT1164 to identify CBDB and CBDP.

FIG. 5 represents the normalization of inflammation-induced iNOS expression by NT1164. The figure shows that NT1164 normalises inflammation-induced iNOS expression.

FIG. 6 presents the neuronal viability quantified using MTT [3-(4,5-dimethylthiazol-2-yl-)-2,5-diphenyl-2H-tetrazolium bromide]. The figure shows that NT1164 increases the number of neurons under basal conditions (short term exposure).

FIG. 7 demonstrates that NT1164 stimulates the maturation of immature neurons into healthy cells even without the presence of any glutamate induced insult. The figure shows the effects of NT1164 alone on neurons (no glutamate).

FIG. 8 demonstrates that CBD is toxic in this paradigm while NT1164 is non-toxic and has positive effects on cell number and cell viability. The figure shows that NT1164 does not increase cell death in an excitotoxic cell injury paradigm.

FIG. 9 shows the microglial responses under inflammatory conditions assessing arginase 1 expressions. The figure shows that NT1164 normalises inflammation-induced (injured cells) Arg1 expression.

FIG. 10 is a diagram which outlines the arginine metabolism and the effects it has on the overall balance of anti-inflammatory and pro-inflammatory signals. (Reference: Review. Gonçalo S. Clemente, Aren van Waarde, Inês F. Antunes, Alexander Dömling and Philip H. Elsinga. Arginase as a Potential Biomarker of Disease Progression: A Molecular Imaging Perspective. (2020)).

FIG. 11 shows the distribution of patients actively using NT1164 for Example 6.

FIG. 12 show the distribution of the severity of illness of active patients at baseline as per CGI-S severity of illness for Example 6.

FIG. 13 shows the maximum tolerated dose for active patients for Example 6.

FIG. 14 shows the CGI-S global improvement at 28 days of NT1164 treatment.

FIG. 15 shows the CGI-S severity of illness after 28 days of treatment.

FIG. 16 shows the CGI-S severity of illness after 28 days of treatment.

FIG. 17 shows the CGI-S therapeutic effect after 28 days of treatment.

FIG. 18 shows the age distribution of patients actively using NT1164 for Example 7.

FIG. 19 show the distribution of the severity of illness of active patients at baseline as per CGI-S severity of illness for Example 7. CGI-S refers to the Clinical Global Impression Scale—Severity of Illness.

FIG. 20 shows the CGI-S global improvement at 20 weeks of NT1164 treatment.

FIG. 21 shows the CGI-S global improvement over time up to and including 20 weeks of NT1164 treatment.

FIG. 22 shows the CGI-S severity of illness at 20 weeks of treatment.

FIG. 23 shows the CGI-S severity of illness over time up to and including 20 weeks of treatment.

FIG. 24 shows the CGI-S severity of illness at 20 weeks of treatment.

FIG. 25 shows the CGI-S therapeutic effect over time up to and including 20 weeks of treatment.

FIG. 26 shows the CGI-S therapeutic effect at 20 weeks of treatment.

### DETAILED DESCRIPTION OF THE INVENTION

For convenience, the following sections generally outline the various meanings of the terms used herein. Following this discussion, general aspects regarding compositions, use of medicaments and methods of the invention are discussed, followed by specific examples demonstrating the properties of various embodiments of the invention and how they can be employed.

Those skilled in the art will appreciate that the invention described herein is susceptible to variations and modifications other than those specifically described. The invention includes all such variations and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness. None of the cited material or the information contained in that material should, however be understood to be common general knowledge.

Manufacturer's instructions, descriptions, product specifications, and product sheets for any products mentioned herein or in any document incorporated by reference herein, are hereby incorporated herein by reference, and can be employed in the practice of the invention.

The present invention is not to be limited in scope by any of the specific embodiments described herein. These embodiments are intended for the purpose of exemplification only. Functionally equivalent products, formulations and methods are clearly within the scope of the invention as described herein.

### 1. Definitions

The meaning of certain terms and phrases used in the specification, examples, and appended claims, are provided below. If there is an apparent discrepancy between the usage of a term in the art and its definition provided herein, the definition provided within the specification shall prevail.

Other than in the operating examples, or where otherwise indicated, all numbers expressing quantities of ingredients or reaction conditions used herein should be understood as modified in all instances by the term “about.” The term “about” when used in connection with percentages can mean±1%.

The invention described herein may include one or more range of values (e.g., size, concentration etc.). A range of values will be understood to include all values within the range, including the values defining the range, and values adjacent to the range which lead to the same or substantially the same outcome as the values immediately adjacent to that value which defines the boundary to the range. For example, a person skilled in the field will understand that a 10% variation in upper or lower limits of a range can be totally appropriate and is encompassed by the invention. More particularly, the variation in upper or lower limits of a range will be 5% or as is commonly recognised in the art, whichever is greater.

In this application, the use of the singular also includes the plural unless specifically stated otherwise. In this application, the use of ‘or means’ and/or unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise. Also, the use of the term “portion” can include part of a moiety or the entire moiety.

Throughout this specification, unless the context requires otherwise, the word “comprise” or variations such as “comprises” or “comprising”, will be understood to imply the inclusion of a stated integer or group of integers but not the exclusion of any other integer or group of integers.

“Therapeutically effective amount” as used herein with respect to methods of treatment and in particular drug dosage, shall mean that dosage that provides the specific pharmacological response for which the drug is administered in a significant number of subjects in need of such treatment. It is emphasized that “therapeutically effective amount,” administered to a particular subject in a particular instance will not always be effective in treating the diseases described herein, even though such dosage is deemed a “therapeutically effective amount” by those skilled in the art. It is to be further understood that drug dosages are, in particular instances, measured as oral dosages, or with reference to drug levels as measured in blood. Amounts effective for such a use will depend on: the desired therapeutic effect; the potency of the biologically active material; the desired duration of treatment; the stage and severity of the disease being treated; the weight and general state of health of the patient; and the judgment of the prescribing physician. Treatment dosages need to be titrated to optimize safety and efficacy. One skilled in the art will appreciate that the appropriate dosage levels for treatment will thus vary depending, in part, upon the indication for which the active agent is being used, the route of administration, and the size (body weight, body surface or organ size) and condition (the age and general health) of the patient. Accordingly, the clinician may titre the dosage and modify the route of administration to obtain the optimal therapeutic effect. A typical dosage may range from about 0.1 μg/kg to up to about 100 mg/kg or more, depending on the factors mentioned above. In other embodiments, the dosage may range from 0.1 μg/kg up to about 100 mg/kg; or 1 μg/kg up to about 100 mg/kg; or 5 μg/kg up to about 100 mg/kg.

The frequency of dosing will depend upon the pharmacokinetic parameters of the active agent and the formulation used. Typically, a clinician will administer the composition until a dosage is reached that achieves the desired effect. The composition may therefore be administered as a single dose, or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. Appropriate dosages may be ascertained through use of appropriate dose-response data.

As used herein “pharmaceutically acceptable carrier” includes any and all solvents, dispersion media, coatings, antibacterial and antifungal agents, isotonic and absorption delaying agents, and the like that are physiologically compatible.

As used herein the term “subject” generally includes mammals such as: humans; farm animals such as sheep, goats, pigs, cows, horses, llamas; companion animals such as dogs and cats; primates; birds, such as chickens, geese and ducks; fish; and reptiles. The subject is preferably human.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

Features of the invention will now be discussed with reference to the following non-limiting description and examples.

### 2. Embodiments

### Composition

The present invention provides a composition comprising the following cannabinoids:
about 50 w/w % of CBDA; and
wherein all other cannabinoids come to about 15 w/w %.

In a preferred embodiment, the invention provides a composition comprising the following cannabinoids:
w/w % about 50% of CBDA; and
about 2% CBD.

In a further preferred embodiment, the invention provides a composition comprising the following cannabinoids:
w/w %
about 50% of CBDA; and
about 5% CBG.

In a further preferred embodiment, the invention provides a composition comprising the following cannabinoids:
w/w %
about 50% of CBDA; and
about 2% CBDP.

In a further preferred embodiment, the invention provides a composition comprising the following cannabinoids:
w/w %
about 50% of CBDA; and
about 2% CBDB.

In a further preferred embodiment, the invention provides a composition comprising the following cannabinoids:
w/w %
about 50% of CBDA; and
about 5% CBGA.

In a further preferred embodiment, the invention provides a composition comprising cannabinoids, wherein the ratio of CBDA to all other cannabinoids is between 4:1 and 2:1.

In a further preferred embodiment, the invention provides a composition comprising cannabinoids, wherein the ratio of CBDA to all other cannabinoids is about 3:1.

In a further preferred embodiment, the invention provides a composition comprising cannabinoids, wherein the ratio of CBDA to all other cannabinoids is about 3.21:1.

In a further preferred embodiment, the invention provides a composition comprising the following cannabinoids:
w/w %
CBDA 40-60%;
CBD 1-5%;
CBG 1-10%;
CBDP 1-5%;
CBDB 1-5%;
CBGA 1-10%;
CBN 1-3%; and
THC<1%.

In a further preferred embodiment, the invention provides a composition comprising the following cannabinoids:
w/w %
CBDA 45-55%;
CBD 1-3%;
CBG 3-7%;
CBDP 1-3%;
CBDB 1-3%;
CBGA 3-7%;
CBN 1-3%; and
THC<0.5%.

In a further preferred embodiment, the invention provides a composition comprising the following cannabinoids:
w/w %
CBDA 50%;
CBD 2%;
CBG 5%;
CBDP 2%;
CBDB 2%;
CBGA 5%;
CBN 3%; and
THC<0.3%.

In a further preferred embodiment, the invention provides a composition comprising the following cannabinoids:
w/w %
CBDA 49%;
CBD 2%;
CBG 5%;
CBDP 2%;
CBDB 2%;
CBGA 5%;
CBN 2%; and
THC<0.3%.

In a further preferred embodiment, the invention provides a composition comprising the following cannabinoids:
w/w %
CBDA 48.78%;
CBD 1.89%;
CBG 4.88%;
CBDP 1.68%;
CBDB 1.76%;
CBGA 4.76%;
CBN 1%; and
THC<0.18%.

In a further preferred embodiment, the invention provides a composition comprising the following cannabinoids:
w/w %
CBDA 45%;
CBD 1%;
CBG 4%;
CBDP 1%;
CBDB 2%;
CBGA 4%;
CBN 2%; and
THC<0.2%.

In a further preferred embodiment, the invention provides a composition comprising the following cannabinoids:
w/w %
CBDA 45.28%;
CBD 1.39%;
CBG 3.88%;
CBDP 1.18%;
CBDB 1.56%;
CBGA 3.76%;
CBN 1%; and
THC<0.18%.

In a further preferred embodiment, the invention provides a composition comprising the following cannabinoids:
w/w %
CBDA 62.78%;
CBD 5.80%;
CBG 0.44%;
CBGA 1.26%;
CBN 1.98%; and
THC<0.70%.

In a further preferred embodiment, the invention provides a composition comprising the following cannabinoids:
w/w %
CBDA 60.29%;
CBD 5.34%;
CBG 0.39%;
CBGA 1.14%;
CBN 0.85%; and
THC<0.65%.

In a further preferred embodiment, the invention provides a composition wherein the cannabinoids are present in amounts selected from the group consisting of:

Composition 1 comprising
w/w %
CBDA 50%;
CBD 2%;
CBG 5%;
CBDP 2%;
CBDB 2%;
CBGA 5%;
CBN 3%; and
THC<0.3%;
and

Composition 2 comprising
w/w %
CBDA 45%;
CBD 1%;
CBG 4%;
CBDP 1%;
CBDB 2%;
CBGA 4%;
CBN 2%; and
THC<0.2%.

In a further preferred embodiment, the invention provides a composition, wherein the quantity of the cannabinoids is determined by a method selected from the group consisting of: high performance chromatography (HPLC), proton nuclear magnetic resonance spectroscopy (H¹ NMR); and mass spectrometry.

In a further preferred embodiment, the invention provides a composition derived from cannabis plant material.

In a further preferred embodiment, the invention provides a composition wherein the said listed cannabinoids are synthetic.

In a further preferred embodiment, the invention provides a composition wherein the said listed cannabinoids are a mixture of plant derived and synthetic cannabinoids.

In a further preferred embodiment, the invention provides a composition further comprising an oil selected from the group consisting of: a synthetic oil; plant based oil; mineral oil; canola oil; and olive oil.

In a further preferred embodiment, the composition comprises less than 5% w/w terpenes.

In a further preferred embodiment, the composition comprises less than 2% w/w organic plant material.

In a further preferred embodiment, the composition comprises less than 2% w/w of plant phenols.

In a further preferred embodiment, the composition comprises components selected from the group consisting of: flavonoids, proteins, sterols and esters.

In a further preferred embodiment, the composition is substantially pure. Preferably, the purity is determined by a method selected from the group consisting of: high performance chromatography (HPLC), proton nuclear magnetic resonance spectroscopy (H¹NMR); and mass spectrometry. Preferably, the purity is selected from the group consisting of: greater than 75% purity; greater than 80% purity; greater than 85% purity; greater than 90% purity; greater than 95% purity; greater than 96% purity; greater than 97% purity; greater than 98% purity; greater than 99% purity; greater than 99.5% purity; greater than 99.6% purity; greater than 99.7% purity; greater than 99.8% purity; greater than 99.9% purity; greater than 99.95% purity; greater than 99.96% purity; greater than 99.97% purity; greater than 99.98% purity and greater than 99.99% purity.

In a further preferred embodiment, the composition comprises less than 0.1 wt % organic impurities as measured a method selected from the group consisting of: high performance chromatography (HPLC), proton nuclear magnetic resonance spectroscopy (H¹ NMR); and mass spectrometry.

In a further preferred embodiment, the composition is substantially free of atmospheric oxygen.

In a further preferred embodiment, the composition is sterile. In an alternative preferred embodiment, the composition is not sterile.

In a further preferred embodiment, the invention provides a composition wherein the cannabinoid component of the composition is selected from the group consisting of: between 1 and 500 mg/ml; between 10 and 100 mg/ml; be at a concentration of 50 mg/ml.

In a further preferred embodiment, the invention provides a composition wherein the CBDA component of the composition is selected from the group consisting of: between 1 and 500 mg/ml; between 10 and 100 mg/ml; be at a concentration of 50 mg/ml.

In a further preferred embodiment, the composition is a liquid.

In a further preferred embodiment, the composition is an oil.

In a further preferred embodiment, the composition demonstrates no cannabinoid degradation or decarboxylation when measured at a time point selected from the group consisting of: at 1 day; at 2 days; at 7 days; at 14 days; at 28 days; at 5 weeks; at 6 weeks; and 32 weeks.

In a further preferred embodiment, the composition demonstrates cannabinoid stability when measured at a time point selected from the group consisting of: at 1 day; at 2 days; at 7 days; at 14 days; at 28 days; at 5 weeks; at 6 weeks and 32 weeks.

In a further preferred embodiment, the composition demonstrates no mutagenicity, carcinogenicity or genotoxicity when delivered at a concentration that delivers 120 mg/ml of CBDA.

In a further preferred embodiment, the composition is adapted to suppress the activity of any one of the following biomarkers: COX-2; iNOS; TNF-alpha; IL-2; IL-12 and GS-MCF.

Preferably, the composition is adapted to suppress neuroinflammation. More preferably, the composition is adapted for the treatment of a neurological disorder.

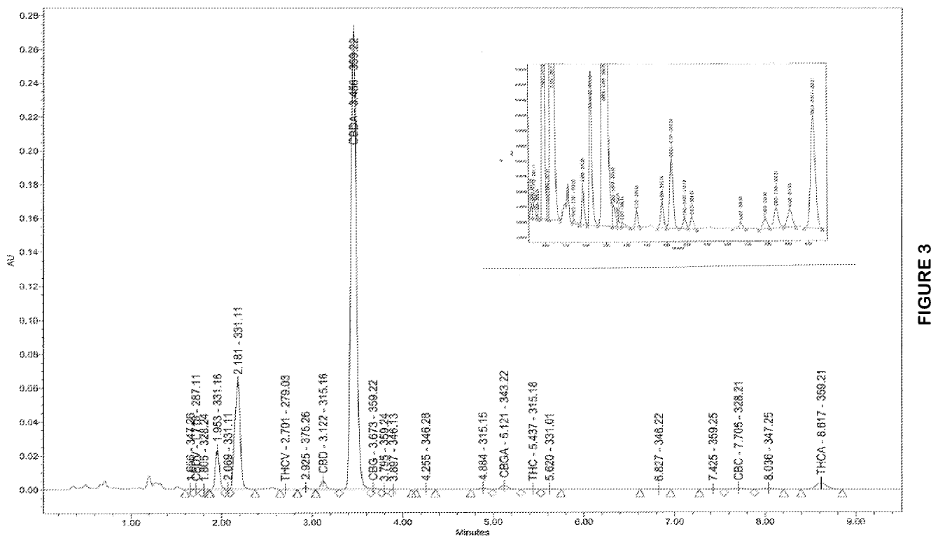

In a further preferred embodiment, the invention provides a composition having a UPLC mass chromatogram corresponding to FIG. 3 utilising the conditions described in Example 1.

In a further preferred embodiment, the composition comprises an additional active ingredient.

Preferably, the additional active ingredient is selected from the group consisting of: a polypeptide; an antibody; a NSAID; a neuroregulator; and a neurotransmitter.

In a further preferred embodiment, the ratio of cannabinoid component and the additional active ingredient is selected from the group consisting of: 1 unit w/w of cannabinoid: 1 unit w/w/ of the additional active ingredient; 2:1; 3:1; 4:1; 5:1; between 10,000:1 and 1:1; between 1,000:1 and 1:1; between 500:1 and 1:1; between 100:1 and 1:1; between 50:1 and 1:1; and between 10:1 and 1:1.

In a further preferred embodiment, the ratio of the additional active ingredient and cannabinoid is selected from the group consisting of: 1 unit w/w of the additional active ingredient and 1 unit w/w/ of the cannabinoid; 2:1; 3:1; 4:1; 5:1; between 10,000:1 and 1:1; between 1,000:1 and 1:1; between 500:1 and 1:1; between 100:1 and 1:1; between 50:1 and 1:1; and between 10:1 and 1:1.

In a further preferred embodiment, the ratio of CBDA and the additional active ingredient is selected from the group consisting of: 1 unit w/w of CBDA:1 unit w/w/ of the additional active ingredient; 2:1; 3:1; 4:1; 5:1; between 10,000:1 and 1:1; between 1,000:1 and 1:1; between 500:1 and 1:1; between 100:1 and 1:1; between 50:1 and 1:1; and between 10:1 and 1:1.

In a further preferred embodiment, the ratio of the additional active ingredient and CBDA is selected from the group consisting of: 1 unit w/w of the additional active ingredient and 1 unit w/w/ of the CBDA; 2:1; 3:1; 4:1; 5:1; between 10,000:1 and 1:1; between 1,000:1 and 1:1; between 500:1 and 1:1; between 100:1 and 1:1; between 50:1 and 1:1; and between 10:1 and 1:1.

In one preferred embodiment, the neuroregulator is a psychedelic substance.

Preferably, the neuroregulator is selected from the group consisting of: 3,4-methylenedioxymethamphetamine; lysergic acid diethylamide; and psilocybin.

In a further preferred embodiment, the composition demonstrates synergistic biological activity.

In a further preferred embodiment, the composition demonstrates a level of biological activity that is greater than the sum of: (1) the biological activity of the cannabinoid component when delivered in absence of the additional active ingredient; and (2) the biological activity of the additional active ingredient when delivered in absence of the cannabinoid component.

In a further preferred embodiment, the biological activity is selected from the group consisting of: suppressing inflammation; suppressing neuroinflammation; treating a neurological disorder; suppressing the activity of COX-2; suppressing the activity of iNOS; suppressing the activity of TNF-alpha; suppressing the activity of IL-2; suppressing the activity of IL-12 and suppressing the activity of GS-MCF.

In a further preferred embodiment, the composition is selected from the group consisting of: a therapeutic composition; a pharmaceutical composition; a cosmetic composition; and a veterinary composition.

### Pharmaceutical Compositions

The present invention also provides a pharmaceutical composition comprising the composition of the invention together with a pharmaceutically acceptable carrier.

Therapeutic compositions are within the scope of the present invention. Preferably the compositions are combined with a pharmaceutically acceptable carrier or diluent to produce a pharmaceutical composition (which may be for human or animal use). Suitable carriers and diluents include isotonic saline solutions, for example phosphate-buffered saline. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions. See, e.g., Remington's Pharmaceutical Sciences, 19th Ed. (1995, Mack Publishing Co., Easton, Pa.) which is herein incorporated by reference.

The pharmaceutical composition can contain formulation materials for modifying, maintaining or preserving, for example, the pH, osmolarity, viscosity, clarity, colour, isotonicity, odour, sterility, stability, rate of dissolution or release, adsorption or penetration of the composition. Suitable formulation materials include, but are not limited to, amino acids (such as glycine, glutamine, asparagine, arginine or lysine); antimicrobials; antioxidants (such as ascorbic acid, sodium sulphite or sodium hydrogen-sulphite, Vitamin E, Vitamin E phosphate—lipid soluble vitamins, nano emulsions); buffers (such as borate, bicarbonate, tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin), fillers; monosaccharides, disaccharides; and other carbohydrates (such as glucose, mannose, or dextrins); proteins (such as serum albumin, gelatin or immunoglobulins); colouring, flavouring (natural and natural derived products) and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (including artificial sweetners such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapol); stability enhancing agents (sucrose or sorbitol); tonicity enhancing agents (such as alkali metal halides, preferably sodium or potassium chloride), delivery vehicles, diluents, excipients and/or pharmaceutical adjuvants.

The optimal pharmaceutical composition will be determined by one skilled in the art depending upon, for example, the intended route of administration, delivery format, and desired dosage. Such compositions may influence the physical state, stability, rate of in vivo release, and rate of in vivo clearance of the composition of the invention. The preferred form of the pharmaceutical composition depends on the intended mode of administration and therapeutic application.

The primary vehicle or carrier in a pharmaceutical composition is aqueous and non-aqueous in nature. For example, a suitable vehicle or carrier may be water for injection, physiological saline solution, possibly supplemented with other materials. Neutral buffered saline or saline mixed with serum albumin are further exemplary vehicles. Other exemplary pharmaceutical compositions comprise tris buffer of about pH 7.0-8.5, or acetate buffer of about pH 4.0-5.5, which may further include sorbitol or a suitable substitute therefor. In one embodiment of the present invention, pharmaceutical compositions may be prepared for storage by mixing the selected composition having the desired degree of purity with optional formulation agents in the form an aqueous solution.

The formulation components are present in concentrations that are acceptable to the site of administration. For example, buffers are used to maintain the composition at physiological pH or at a slightly lower pH, typically within a pH range of from about 5 to about 8.

Additional pharmaceutical compositions will be evident to those skilled in the art, including formulations of the invention in sustained- or controlled-delivery formulations. Techniques for formulating a variety of other sustained- or controlled-delivery means, such as liposome carriers, bio-erodible microparticles or porous beads and depot injections, are also known to those skilled in the art. Additional examples of sustained-sustained-release preparations include semipermeable polymer matrices in the form of shaped articles, for example, films, or microcapsules. Sustained release matrices may include polyesters, hydrogels, polylactides, copolymers of L-glutamic acid and gamma ethyl-L-glutamate, ethylene vinyl acetate or poly-D(−)-3-hydroxybutyric acid. Sustained-release compositions may also include liposomes, which can be prepared by any of several methods known in the art.

The pharmaceutical composition to be used for in vivo administration typically must be sterile. This may be accomplished by filtration through sterile filtration membranes. In addition, the compositions generally are placed into a container having a sterile access port. Once the pharmaceutical composition has been formulated, it may be stored in sterile vials as a solution.

In yet a further preferred embodiment, the composition retains its effective biological activity for a period selected from the group consisting of; greater than 24 hours; greater than 36 hours; and greater than 48 hours. Preferably, the composition is stable for periods selected from the group consisting of: 6 months, 1 year and 2 years. In one example, the composition is stable at temperatures selected from the group consisting of: −4° C., 4° C., 18° C. and 25° C.

### Dosage Form

Dosage forms are within the scope of the invention. In a preferred embodiment, the invention provides a dosage form comprising the composition as described in the first aspect of this invention.

Preferably, the cannabinoid component of the composition of the dosage form is selected from the group consisting of: between 1 mg and 1000 mg; between 1 mg and 500 mg; between 1 and 100 mg; less than 400 mg; less than 300 mg; less than 200 mg and less than 100 mg. More preferably the cannabinoid component of the composition is selected from the group consisting of: 600 mg; 400 mg; 300 mg; 200 mg; 100 mg; 50 mg; 10 mg; 5 mg; 2 mg; 1 mg. Preferably the CBDA component of the composition of the dosage form is selected from the group consisting of: between 1 mg and 1000 mg; between 1 mg and 500 mg; between 1 and 100 mg; less than 400 mg; less than 300 mg; less than 200 mg and less than 100 mg. More preferably, the CBDA component of the composition is selected from the group consisting of: 600 mg; 400 mg; 300 mg; 200 mg; 100 mg; 50 mg; 10 mg; 5 mg; 2 mg; 1 mg.

In a further embodiment, the dosage form is form selected from the group consisting of: a solution, tablet, capsule, wafer, dry power sachet and vial/freeze dried.

Preferably, the dosage form is stored in a sealed and sterile container.

### Method for Treating

The invention also provides a method of treating a disorder, said method comprising administering to a patient in need thereof a therapeutically effective amount of the dosage form of the invention.

In a further preferred embodiment, the dosage form is administered at an amount to at least partially treat the disorder.

In a further preferred embodiment, the therapeutically effective amount is an amount of cannabinoid selected from the group consisting of: between 1 to 100 mg/kg/day; between 2 and 50 mg/kg/day; between 5 and 40 mg/kg/day; between 10 and 30 mg/kg/day; between 20 and 25 mg/kg/day; and 20 mg/kg/day. Preferably, the therapeutically effective amount is an amount of cannabinoid vis selected from the group consisting of: 10 mg/day; 15 mg/day; 40 mg/day; 400 mg/day; 600 mg/day; 800 mg/day; 1280 mg/day; 1500 mg/day.

In a further preferred embodiment, therapeutically effective amount is an amount of CBDA selected from the group consisting of: between 1 to 100 mg/kg/day; between 2 and 50 mg/kg/day; between 5 and 40 mg/kg/day; between 10 and 30 mg/kg/day; between 20 and 25 mg/kg/day; and 20 mg/kg/day. Preferably, the therapeutically effective amount is an amount of CBDA vis selected from the group consisting of: 10 mg/day; 15 mg/day; 40 mg/day; 400 mg/day; 600 mg/day; 800 mg/day; 1280 mg/day; 1500 mg/day.

In a further preferred embodiment, Tmax occurs between 1 and 4 hours.

In a further preferred embodiment, T½ occurs between 1.1 and 2.4 hours.

In a further preferred embodiment, the therapeutically effective amount is administered to the subject to treat the disorder.

Preferably the therapeutically effective amount is administered to the subject utilising a dosing regimen selected from the group consisting of: twice hourly; hourly; once every six hours; once every 8 hours; once every 12 hours; once daily; twice weekly; once weekly; once every 2 weeks; once every 6 weeks; once a month; every 2 months; every 3 months; once every 6 months; and once yearly.

Preferably the therapeutically effective amount is administered to the subject using a method selected from the group consisting of: orally, intravenously, intramuscularly, intrathecally, subcutaneously, sublingually, buccally, rectually, vaginally, topically, parentally, mucosally, by the ocular route, by the otic route, nasally, by inhalation, cutaneously, transdermally, and systemically.

In a further preferred embodiment, the disorder is caused by inflammation.

In a further preferred embodiment, the disorder is caused by neuro-inflammation.

Preferably, the disorder is a neurological disorder. More preferably, the neurological disorder is selected from the group consisting of: Alzheimer's disease; Parkinson's disease; multiple sclerosis; amyotrophic lateral sclerosis; cerebral ischemia; traumatic brain injury; rheumatoid arthritis; chronic migraine; epilepsy; autism spectrum disorder; attention deficit hyperactivity disorder; cerebral palsy and relevant subtypes; neuropathic pain; and depression.

In a further preferred embodiment, the ASD is ASD Level II/III and being either ‘Mildly ill’, ‘Moderately ill’, ‘Markedly ill’ or ‘Severely ill’ on the CGI Severity scale.

In a further preferred embodiment, the treatment reduces the neuro-inflammation. Preferably, the treatment suppresses the activity of any one of the following biomarkers: COX-2; iNOS; TNF-alpha; IL-2; IL-12 and GS-MCF.

A subject that can be treated with the invention will include humans as well as other mammals and animals.

In a further preferred embodiment, the method comprises administering to a patient in need thereof a therapeutically effective amount of the dosage form of the invention together with an additional active ingredient. In a preferred form, the additional active ingredient is administered using a dosing regimen selected from the group consisting of: at the same time as administering the dosing form of the invention; before administering the dosing form of the invention; after administering the dosing form of the invention; concurrently with administering the dosing form of the invention; sequentially before administering the dosing form of the invention; and sequentially after administering the dosing form of the invention.

The effect of the administered therapeutic composition can be monitored by standard diagnostic procedures.

### Use of a Composition in the Manufacture of a Medicament

Uses are within the scope of this invention. The invention also provides a use of the composition of the first aspect of the invention in the manufacture of a medicament for the treatment of a disorder.

In one preferred embodiment, the invention is the use of a composition comprising the following cannabinoids:
w/w %
CBDA 40-60%;
CBD 1-5%;
CBG 1-10%;
CBDP 1-5%;
CBDB 1-5%;
CBGA 1-10%;
CBN 1-3% and THC<1%;
in the manufacture of a medicament for the treatment of a disorder.

In a further embodiment, the cannabinoids of the composition are present in amounts selected from the group consisting of:

Composition 1 comprising
w/w %
CBDA 50%;
CBD 2%;
CBG 5%;
CBDP 2%;
CBDB 2%;
CBGA 5%;
CBN 3% and
THC<0.3%;
and

Composition 2 comprising
w/w %
CBDA 45%;
CBD 1%;
CBG 4%;
CBDP 1%;
CBDB 2%;
CBGA 4%;
CBN 2% and
THC<0.2%;
in the manufacture of a medicament for the treatment of a disorder.

In a further embodiment, the composition further comprises an oil selected from the group consisting of: a synthetic oil; plant based oil; mineral oil; canola oil; and olive oil.

In a further embodiment, the composition comprises less than 5% w/w terpenes.

In a further embodiment, the composition comprises less than 2% w/w organic plant material.

In a further embodiment, the composition comprises less than 2% w/w of plant phenols.

In a further embodiment, the cannabinoid component of the composition is selected from the group consisting of: between 1 and 500 mg/ml; between 10 and 100 mg/ml; be at a concentration of 50 mg/ml.

In a further embodiment, the CBDA component of the composition is selected from the group consisting of: between 1 and 500 mg/ml; between 10 and 100 mg/ml; be at a concentration of 50 mg/ml.

In a further embodiment, the composition has a UPLC mass chromatogram corresponding to FIG. 3 utilising the conditions described in Example 1.

### Processes

The invention also provides a process of extracting the composition of the first aspect of the invention from cannabis plant material, said process comprising the steps of:
1) Grinding the cannabis plant material to a sufficient grind size;
2) Contacting the grind produced by step a) with oil;
3) Mixing the grind and oil for a sufficient time period to form a mixture;
4) Pressing the mixture to reclaim the oil;
5) Centrifuging the oil to further refine the oil; and
6) Collecting the oil extract in a suitable container/steel vessel.

In a further preferred embodiment, the cannabis plant material is derived from Cannabis sativa L.

In a further preferred embodiment, the sufficient grind size is selected from the group consisting of: between 0.1 mm and 3 mm; between 1 mm and 2 mm; and between 0.5 mm and 2.5 mm.

In a further preferred embodiment, the sufficient time period is selected from the group consisting of: between 30 minutes and 2 hours; between 45 minute and 1.5 hours; 1 hr.

In a further preferred embodiment, the ratio of grind material to oil at step (2) is selected from the group consisting of: 400 mg of grind: 1 ml of oil; 300 mg of grind: 1 ml of oil; 200 mg of grind: 1 ml of oil; 100 mg of grind: 1 ml of oil; and 333 mg of grind: 1 ml of oil.

Preferably, the oil is olive oil.

The invention also provides an alternative process of extracting the composition of the first aspect of the invention from cannabis plant material, said process comprising the steps of:
1) Grinding the cannabis plant material to a sufficient grind size;
2) Contacting the grind produced by step a) with an alcohol;
3) Mixing the grind and alcohol for a sufficient time period to form a mixture;
4) Sonicating the mixture;
5) Centrifuging the mixture; and
6) Collecting the alcohol extract in a suitable container.

In a further preferred embodiment, the alcohol is ethanol.

In a further preferred embodiment, the alcohol is selected from the group consisting of: ethanol, isopropyl alcohol, methyl alcohol, benzyl alcohol, 1,4-butanediol, 1,2,4-butanetriol, butanol, 1-butanol, 2-butanol, and tert-butyl alcohol.

In a further preferred embodiment, the sufficient grind size is selected from the group consisting of: between 0.1 mm and 3 mm; between 1 mm and 2 mm; and between 0.5 mm and 2.5 mm. In a further preferred embodiment, the sufficient time period is selected from the group consisting of: between 30 minutes and 2 hours; between 45 minute and 1.5 hours; 1 hr.

In a further preferred embodiment, the ratio of grind material to alcohol at step (2) is selected from the group consisting of: 400 mg of grind: 1 ml of alcohol; 300 mg of grind: 1 ml of alcohol; 200 mg of grind: 1 ml of alcohol; 100 mg of grind: 1 ml of alcohol; 100 mg of grind: 4 ml of alcohol; 100 mg of grind: 3 ml of alcohol; 100 mg of grind: 2 ml of alcohol; and 333 mg of grind: 1 ml of alcohol.

### Product of the Process

The invention also provides a product produced from the process described above.

The invention also provides a kit comprising the dosage form of one aspect of the invention together with instructions for its use.

### Device

Devices are within the scope of the invention. In a preferred embodiment, the invention provides a device, wherein the device comprises: (1) the composition as described in the first aspect of this invention; and (2) an applicator.

### Method for Stabilising

Methods for stabilizing the composition are within the scope of the invention.

In a further preferred embodiment, the said method protects the composition against degradation.

In yet a further preferred embodiment, the composition retains its effective biological activity for a period selected from the group consisting of; greater than 24 hours; greater than 36 hours; greater than 48 hours.

The addition of approved pharmaceutical excipients to stabilise the composition is preferred from a safety standpoint, as the simpler methodology is likely to produce a less variable outcome and the choice of excipient can be limited to those with Generally Regarded as Safe (GRAS) status. Excipients for the stabilisation of protein solutions can be classified into four broad categories: salts, sugars, polymers or protein/amino acids, based on their chemical properties and mechanism of action. Salts (e.g., chlorides, nitrates) stabilise the tertiary structure of proteins by shielding charges through ionic interactions. Sugars (e.g., glycerol, sorbitol, fructose, trehalose) increase the surface tension and viscosity of the solution to prevent protein aggregation. Similarly, polymers (e.g. polyethylene glycol, cellulose derivatives) stabilise the protein tertiary structure by increasing the viscosity of the solution to prevent protein aggregation and intra- and inter-molecular electrostatic interactions between amino acids in the protein. Proteins (e.g. human serum albumin) are able to stabilise the structure of other proteins through ionic, electrostatic and hydrophobic interactions. Similarly, small amino acids with no net charge, such as alanine and glycine, stabilise proteins through the formation of weak electrostatic interactions.

As discussed above, the medicaments of the present invention may include one or more pharmaceutically acceptable carriers. The use of such media and agents for the manufacture of medicaments is well known in the art. Except insofar as any conventional media or agent is incompatible with the pharmaceutically acceptable material, use thereof in the manufacture of a pharmaceutical composition according to the invention is contemplated. Pharmaceutical acceptable carriers according to the invention may include one or more of the following examples:
a. surfactants and polymers, including, however not limited to polyethylene glycol (PEG), polyvinylpyrrolidone, polyvinylalcohol, crospovidone, polyvinylpyrrolidone-polyvinylacrylate copolymer, cellulose derivatives, HPMC, hydroxypropyl cellulose, carboxymethylethyl cellulose, hydroxypropylmethyl cellulose phthalate, polyacrylates and polymethacrylates, urea, sugars, polyols, and their polymers, emulsifiers, sugar gum, starch, organic acids and their salts, vinyl pyrrolidone and vinyl acetate; and/or
b. binding agents such as various celluloses and cross-linked polyvinylpyrrolidone, microcrystalline cellulose; and/or (3) filling agents such as lactose monohydrate, lactose anhydrous, microcrystalline cellulose and various starches; and/or
c. filling agents such as lactose monohydrate, lactose anhydrous, mannitol, microcrystalline cellulose and various starches; and/or
d. lubricating agents such as agents that act on the increased ability of the dosage form to be ejected from the packaging cavity, and/or
e. sweeteners such as any natural or artificial sweetener including sucrose, xylitol, sodium saccharin, cyclamate, aspartame, and acesulfame K; and/or
f. flavouring agents; and/or
g. preservatives such as potassium sorbate, methylparaben, propylparaben, benzoic acid and its salts, other esters of parahydroxybenzoic acid such as butylparaben, alcohols such as ethyl or benzyl alcohol, phenolic chemicals such as phenol, or quarternary compounds such as benzalkonium chloride; and/or
h. buffers; and/or
i. diluents such as pharmaceutically acceptable inert fillers, such as microcrystalline cellulose, lactose, dibasic calcium phosphate, saccharides, and/or mixtures of any of the foregoing; and/or
j. absorption enhancer such as glyceryl trinitrate; and/or
k. other pharmaceutically acceptable excipients.

Medicaments of the invention suitable for use in animals and in particular in human beings typically must be sterile and stable under the conditions of manufacture and storage.

The invention also provides a composition, methods and processes as described by the foregoing examples.

The present invention will now be described with reference to the following non-limiting Examples. The description of the Examples is in no way limiting on the preceding paragraphs of this specification, however, is provided for exemplification of the methods and compositions of the invention.

### EXAMPLES

It will be apparent to persons skilled in the milling and pharmaceutical arts that numerous enhancements and modifications can be made to the above-described processes without departing from the basic inventive concepts. For example, in some applications the biologically active material may be pretreated and supplied to the process in the pretreated form. All such modifications and enhancements are considered to be within the scope of the present invention, the nature of which is to be determined from the foregoing description and the appended claims. Furthermore, the following Examples are provided for illustrative purposes only, and are not intended to limit the scope of the processes or compositions of the invention.

### A Example 1—Extraction and Purification of NTI164

### A.1 Study Aim

To extract and identify the most desirable components from the NT1164 plant strain using an inert oil-based extraction process.

### A.2 Materials and Methods

### A.2.1 NT1164 Plant Material

The NT1164 plant is a full-spectrum medicinal cannabis plant (genus species Cannabis sativa) which the inventors subsequently identified to contain cannabidiolic acid (CBDA), cannabidiol (CBD), cannabigerolic acid (CBGA), cannabidivarin (CBDV) and cannabinol (CBN) but which has >0.03% tetrahydrocannabinol (THC). The NT1164 plant was cultivated, dried and packaged under an Office of Drug Control (ODC) license and permit as per Good Manufacturing Processes (GMP) and TGO 93 and 100 guidelines.

### A.2.2 Extraction Method—Oil Based

Equipment: The following equipment was used: 10 mL glass scintillation bottles with lids; Cobram's Estate olive oil; plant grinder (similar to a coffee or food grade grinder) pore size up to 50 μM; Whatman paper, grade 1; pipettes; weight scale (transfer boats and spoons); Eppendorf tubes; 50 mL falcon tubes; bench top centrifuge (Eppendorf Centrifuge 5702); Oz Design Brand 6 Litre Fruit, Wine and Cider Press.

Extraction: Pressing and Centrifugation: All work is undertaken at standard lab temperatures (18-22° C.). The buds of NT1164 were stripped off hard stalks and the stalks discarded. The grinder was cleaned with 70% EtOH and the grinding compartment was filled with dried plant material. The material was ground on the finest of the three setting for 10 seconds (1-2 mm particle size). The grounds were then mixed with 100 ml of olive oil in an autoclaved Schott bottle at a ratio plant/oil of 333 mg/mL. It was then placed on a stirrer at room temperature for 1 hour, stirred with magnetic flea (50 rpm). The oil plus plant mixture is then put into the Oz Design Brand 6 Litre Fruit, Wine and Cider Press to reclaim the oil component from the plant (the mash). The reclaimed oil was then placed into 50 mL falcon tubes and spun at 300 g for 15 minutes at room temperature (Isolation 1). The oil was then removed into a clean Schott bottle and keeping track of the volume reclaimed. The recovery of the oil for Isolation 1 is approximately 40%. The mash is discarded following each isolation. To the reclaimed oil, we added a further 333 mg/mL ground plant/oil (a further 100 ml) material and repeated the 1 hour mix, and reclaimed and re-used oil, until a total of 999 μg/mL (3×100 ml) of plant/oil mixture passed through (Isolation 2). The recovery of the oil for Isolation 2 is approximately 50%. For the final time, we placed into falcon tubes and spin as discussed above (Isolation 3). The recovery of the oil at for Isolation 3 is approximately 50%. We then collected the oil only and placed the oil into Eppendorf tubes for processing. This triplicate extraction method resulted in a total volume of 50 ml of final product at a concentration of 48 mg of CBDA to 1 ml of olive oil determined using UPLC potency testing using the methods described below.

### A.2.3 Extraction Method—Ethanol Based

Extraction: Pressing and Centrifugation: An alternative method includes an extraction based on the use of ethanol. In this method, 500 milligrams of ground plant material of NT1164 is mixed with 20 ml of ethanol in a 50 ml centrifuge tube. The tubes are shaken vigorously for 60 seconds and then placed into a sonicate bath at 30 C for 10 minutes. Samples are then placed on a shaker (200 rpm) for 30 minutes. Once completed, placed in a centrifuge, and centrifuged at 4400 rcf for 5 minutes. The supernatant can then be assessed in the various preclinical models.

### A.2.4 Analytical Analysis

Ultra-performance liquid chromatography (UPLC) reverse-phase and liquid chromatography mass spec (LCMS) were used to identify the components in the NT1164 concentrate derived from the methods discussed above. The analysis was performed using an integrated (U)HPLC system and a single quadrupole mass spectrometer detector with electrospray ionization (ESI) interface.

The UPLC settings and conditions used were: Cortecs UPLC Shield RP 18, (0 A 1.6 uM, 2.1×100 mm); Analytical flow rate: 0.7 ml/min; Mobile phase A: Water 0.1% TFA; Mobile phase B: Acetonitrile; Isocratic: 41:59 mobile phase A/mobile phase B; Temp: 35 C; Detector: Acquity UPLC PDA; Injection volume: 0.7 uL for 1.0 mg/ml reference standard preparations, sample solutions scaled appropriately; Software: Empower 3CDS. Reference standard solutions were obtained from Novachem, Cerilliant Corporation (TX, USA). These were pre-dissolved solutions all previously shown to be suitable for the generation of calibration curve.

A mixture of 16 cannabinoids in methanol was prepared, containing 10 ppm each of cannabidivarin (CBDV), cannabidiol (CBD), cannabigerol (CBG), tetrahydrocannabivarin (THCV), cannabinol (CBN), Δ⁹-tetrahydrocannabinol (Δ⁹-THC), Δ⁸-tetrahydrocannabinol (Δ⁸-THC), cannabichromene (CBC), their respective acidic forms and cannabicyclol (CBL). All solvents used were LCMS grade, and standards were prepared by diluting with 90% mobile phase B and 10% deionized water. Detailed analytical conditions for the UPLC-LCMS analysis are listed in Table 1.

TABLE 1
The parameters and conditions for UPLC and LCMS analysis
HPLC

| | |
|---|---|
| Column + Guard column | NexLeaf CBX II, 1.8 μm, 3.0 × 100 mm |
| Mobile phase A | 0.1% formic acid and 5 mM ammonium |
| | formate in H₂O |
| Mobile phase B | 0.1% formic acid in methanol/ |
| | acetonitrile (50:50 v/v) |
| Gradient | 83% mobile phase B to 98% mobile |
| | phase B in 6.5 min |
| Flow rate | 0.5 mL/min |
| Oven temperature | 30° C. |
| Injection volume | 5 μL |

LCMS

| | |
|---|---|
| Ionization | ESI |
| Interface temperature | 350° C. |
| DL temperature | 250° C. |
| Nebulizing gas flow | 15 L/min |
| Heat block | 400° C. |
| Drying gas flow | 1.5 L/min |
| Q-array DC voltage | 55 V |

### A.3 Results

### A.3.1 UPLC and LCMS Analytical Results

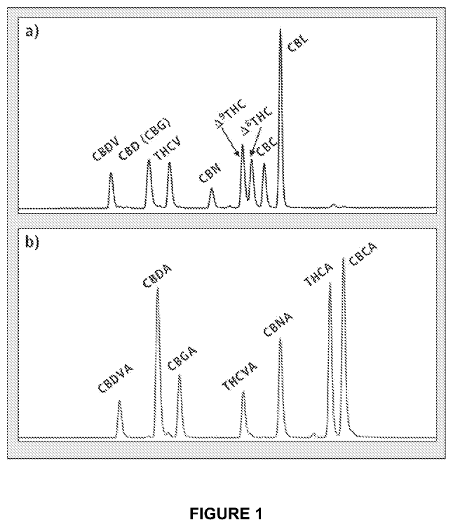

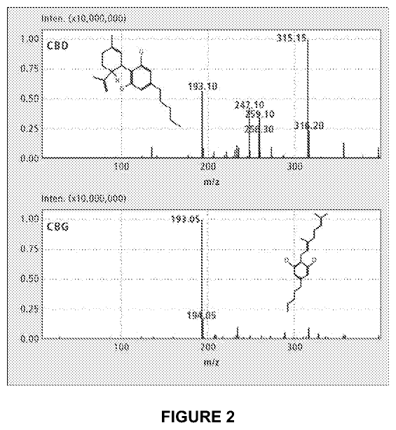

FIG. 1 shows the separation of the cannabinoids in a mixed standard solution (that is a reference solution). Under the conditions of the experiment, neutral cannabinoids such as Δ⁹-THC, CBD and CBL ionize in positive mode, while their respective acidic forms ionize in negative mode. Although CBD and CBG coelute from the column, their molecular weights differ, and they can be identified by mass spectra. In addition, FIG. 2 shows a difference between the SID fragmentation patterns obtained for CBD and CBG (that is; as further reference solution). These highly specific results show the advantage of LCMS over LC-UV for analysis and identification of cannabinoids.

FIG. 3 presents the UPLC mass chromatogram for NT1164 extracted using the oil-based method. These results found that the NT1164 extract (oil suspension) contained the following components presented in Table 2. Additional components will include flavonoids, proteins, phenols, sterols and esters. These are known components that make up 30-40% of the full plant cannabis material. Table 4 presents the accompanying exlution times for the UPLC mass chromatograph for FIG. 3 and area under the peaks for the CBD peaks identified.

TABLE 2
Components in NTI164 oil extracted (at two decimal places
and rounded up beyond 0.5, and rounded down below 0.5)

| Component | w/w % | w/w % |
|---|---|---|
| Cannabidiol acid (CBD-A) | 48.78% | 49% |
| Cannabidiol (CBD) | 1.89%; | 2%; |
| Cannabigerol (CBG) | 4.88%; | 5%; |
| Cannabidiphorol (CBDP) | 1.68%; | 2%; |
| Canabidibutol (CBDB) | 1.76%; | 2%; |
| Cannabigerolic acid (CBGA) | 4.76%; | 5%; |
| Tetrahydrocannabinol (THC) | <0.18% | <0.3% |
| Terpenes | <5% | <5% |
| Organic plant material - including phenols | 2% | 2% |

Table 3 presents the NTI164 composition extracted using the ethanol extraction and the components quantified using the methods herein described.

TABLE 3
Components in NTI164 ethanol extracted (at two decimal places
and rounded up beyond 0.5, and rounded down below 0.5)

| Component | w/w % | w/w % |
|---|---|---|
| Cannabidiol acid (CBD-A) | 45.28% | 45% |
| Cannabidiol (CBD) | 1.39%; | 1%; |
| Cannabigerol (CBG) | 3.88%; | 4%; |
| Cannabidiphorol (CBDP) | 1.18%; | 1%; |
| Canabidibutol (CBDB) | 1.56%; | 2%; |
| Cannabigerolic acid (CBGA) | 3.76%; | 4%; |
| Tetrahydrocannabinol (THC) | <0.18% | <0.2% |
| Terpenes | <5% | <5% |
| Organic plant material - including phenols | 2% | 2% |

Table 4 presents the accompanying elution times for the UPLC mass chromatogram for FIG. 3 (NTI164) and area under the peaks for the CBD peaks identified.

TABLE 4
elution times and area under the peak

| | | | Area under |
|---|---|---|---|
| | | Elution time | the peak |
| Component | w/w % | (min) | (quantity) |
| Cannabidiol (CBD) | 1.89%; | 3.122 | 20876 |
| Cannabidiol acid (CBD-A) | 48.78% | 3.456 | 1161809 |
| Cannabigerol (CBG) | 4.88%; | 3.673 | 16332 |
| Cannabidiphorol (CBDP) | 1.68%; | See comments | See comments |
| Canabidibutol (CBDB) | 1.76%; | See comments | See comments |
| Cannabigerolic acid | 4.76%; | 5.121 | 13492 |
| (CBGA) | | | |
| Tetrahydrocannabinol | <0.18% | 5.437 | 1980 |
| (THC) | | | |
| THCA | <0.18% | 8.617 | 30141 |
| Terpenes | <5% | | |
| Organic plant material - | 2% | | |
| including phenols | | | |

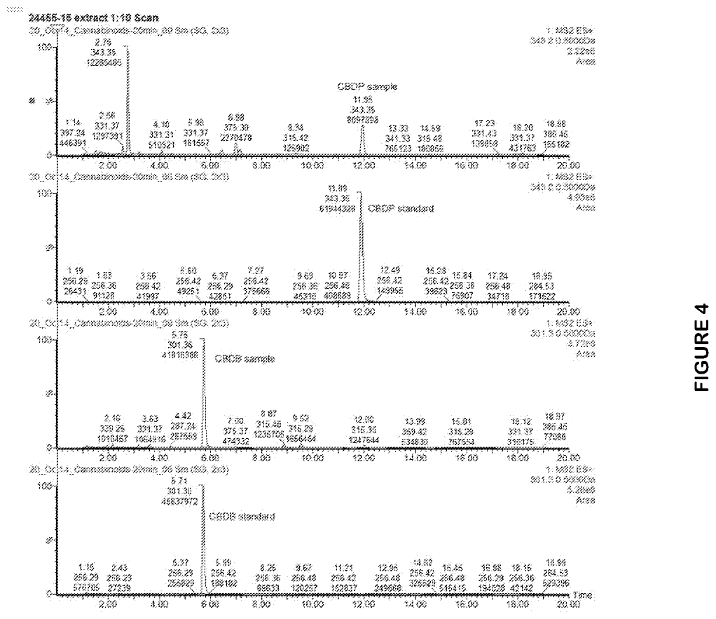

Please note that the rarer cannabinoids such as CBDB and CBDP are only detected using Quad MS (which is different to the routine HPLC used for the other cannabinoids). These results are presented in FIG. 4.

### B Example 2—Characterisation of the Stability Properties of NT1164

### B.1 Study Aim

To assess the stability of the NT1164 samples suspended in oil formulation at room temperature.

### B.2 Materials and Methods

### B.2.1 Sample Preparation

Triplicate samples of NT1164 were prepared using the methods described above.

For control samples, three representative pre-prepared concentrate samples NT1164 (oil and dried flower) were obtained as follows. Oil S=samples were prepared as outlined above: For flower, a portion of homogenized plant material was added to acetonitrile or ethanol and sonicated for 20 minutes. The subsequent extract was filtered through a 0.22 μm syringe tip filter directly into a 2 mL sample vial for analysis. Concentrates were prepared similarly with isopropanol as the extraction solvent.

### B.2.2 Sampling

Samples of NT1164 were assayed on a weekly basis and CBDA was used as a main marker/stability indicator. The ACQUITY UPLC H-Class System combined with the CORTECS UPLC Shield RP18 particle chemistry was used to provide a UPLC isocratic separation of main cannabinoids in a 10.5-minute cycle time. Analytical methods using UPLC were used as described above.

Reference standard solutions were obtained from Cerilliant Corporation (Round Rock, TX). These pre-dissolved solutions have been previously shown to be suitable for the generation of calibration curves.

Preparation of standard curves were performed as follows. Linearity of primary cannabinoids (−)Δ⁹-THC and CBD were determined for 10 concentrations between 0.004 mg/mL and 1.000 mg/mL, prepared via serial dilution in methanol using appropriate standards as a representative demonstration of method linearity. Table 5 outlines the cannabinoids used in the separation.

TABLE 5
The cannabinoids used in the separation

| | | Cannabinoid | |
|---|---|---|---|
| No. | Cannabinoid | abbreviation | CAS number |
| 1 | Delta-9-tetrahydrocannabinol | (−)Δ9-THC | 1972-08-03 |
| 2 | Delta-9-tetrahydrocannabinolic acid | THC-A | 23978-85-0 |
| 3 | Cannabidiol | CBD | 13956-29-1 |
| 4 | Cannabidiol acid | CBD-A | 1244-58-2 |
| 5 | Cannabinol | CBN | 521-35-7 |
| 6 | Delta-8-tetrahydrocannabinol | (−)Δ8-THC | 5957-75-5 |
| 7 | Cannabigerol | CBG | 25654-31-3 |
| 8 | Cannabigerolic acid | CBG-A | 25555-57-1 |
| 9 | Cannabichromene | CBC | 20675-51-8 |
| 10 | Cannabichromenic acid | CBC-A | 185505-15-1 |
| 11 | Tetrahydrocannabivarin | THCV | 31262-37-0 |
| 12 | Tetrahydrocannabivarinic acid | THCV-A | 39986-26-0 |
| 13 | Cannabidivarin | CBDV | 24274-48-4 |
| 14 | Cannabidivarinic acid | CBDV-A | 31932-13-5 |
| 15 | Cannabicyclol | CBL | 21366-63-2 |
| 16 | Cannabicyclolic acid | CBL-A | 40524-99-0 |

### B.3 Results

### B.3.1 UPLC/Mass Spectrometry Analytical Results

The ACQUITY UPLC H-Class System combined with the CORTECS UPLC Shield RP18 particle chemistry was used to provide a UPLC isocratic separation of main cannabinoids in a 10.5-minute cycle time. Samples of NTI164 were assayed on a weekly basis and CBDA was used as a main marker as a stability indicator. Results presented in Table 6 demonstrate that NT1164 is stable at room temperature within an inert oil media over 6 weeks. There is no decarboxylation or product degradation observed over this time frame.

TABLE 6
Stability of NTI164 at room temperature

| | | | Time Point | | | | | | | |
|---|---|---|---|---|---|---|---|---|---|---|
| | | | Day 0 | Day 2 | Day 7 | Day 14 | Day 28 | Week 5 | Week 6 | Week 32 |
| | | | Testing Date | | | | | | | |
| | | | 13 Jul. | 15 Jul. | 20 Jul. | 29 Jul. | 12 Aug. | 19 Aug. | 27 Aug. | 12 Sep. |
| | | | 2021 | 2021 | 2021 | 2021 | 2021 | 2021 | 2021 | 2022 |
| | | | SAMPLE ID | | | | | | | |
| CANNABINOIDS | Cas No's | Units | 28206-7 | 28206-7 | 28206-7 | 28206-7 | 28206-7 | 28206-7 | 28206-7 | 28206-7 |
| Cannabidiol (CBD) | 13956-29-1 | mg/mL | 0.46 | 0.56 | 0.53 | 0.56 | 0.54 | 0.60 | 0.51 | 0.48 |
| Cannabidiol acid (CBD-A) | 1244-58-2 | mg/mL | 48.16 | 48.19 | 48.62 | 48.09 | 47.12 | 48.35 | 47.56 | 47.01 |
| Tetrahydrocannabinol | 1972-08--03 | mg/mL | <0.1 | <0.1 | <0.1 | <0.1 | <0.1 | 0.14 | <0.1 | 0.11 |
| (D9-THC) | | | | | | | | | | |
| (Tetrahydrocannabinolic | 23978-85-0 | mg/mL | 0.66 | 0.72 | 0.64 | 0.69 | 0.36 | 0.79 | 0.73 | <0.1 |
| acid) (D9-THCA) | | | | | | | | | | |
| Cannabidivarin (CBDV) | 24274-48-4 | mg/mL | <0.1 | <0.1 | <0.1 | <0.1 | <0.1 | 0.10 | <0.1 | 0.13 |
| Cannabigerol Acid(CBG-A) | 25555-57-1 | mg/mL | 1.07 | 1.18 | 1.20 | 1.19 | 0.81 | 1.08 | 1.00 | 1.05 |
| Cannabigerol (CBG) | 25654-31-3 | mg/mL | 5.21 | 5.23 | 5.14 | 5.1 | 5.1 | 5.30 | 5.28 | 5.12 |
| Tetrahydrocannabivarin | 28172-17-0 | mg/mL | <0.1 | <0.1 | <0.1 | <0.1 | <0.1 | <0.1 | <0.1 | <0.1 |
| (THCV) | | | | | | | | | | |
| Cannabinol (CBN) | 521-35-7 | mg/mL | 2.32 | 2.54 | 2.48 | 2.33 | 2.56 | 2.22 | 2.27 | 2.21 |
| DELTA-8-THC (D8-THC) | 5957-75-5 | mg/mL | <0.1 | <0.1 | <0.1 | <0.1 | <0.1 | <0.1 | <0.1 | <0.1 |
| Cannabichrome (CBC) | 20675-51-8 | mg/mL | 1.23 | 1.19 | 1.34 | 1.22 | 1.27 | 1.20 | 1.15 | 1.22 |

### C Example 3—Characterisation of the Biological Properties of NT164

### C.1 Study Aim

To assess the anti-inflammatory and neuro-protective effects of NT1164 in neuronal and microglial cell lines.

Neuroinflammation is one of the main triggers of neurodegeneration. Research into the factors and pathways able to induce the first steps of the inflammatory response would lead to the identification of potential therapeutic targets through which to halt the progression of many disorders.

### C.2 Materials and Methods

### C.2.1 Sample Preparation and Dilutions

500 mg of dried plant material of NT1164 is suspended in 20 ml of absolute ethanol (using 50 ml blue top falcon tubes appropriate for centrifugation) and stirred/shaken vigorously for 60 seconds. The tubes are then placed in a sonication bath for 10 mins at 35-40 C. Upon completion of the sonication samples are then placed in a tray shaker (200 rpm) for 30 minutes at room temperature. Once completed the samples are then centrifuged at 4400 rpm for 5 minutes. The supernatant is collected for testing and development.

Units used to describe treatments for test products and concentrations for NT1164.
a. 1/1000 dilution of extract—10 UL (stock material is NT1164—10 UL, which equates to 2 μg/ml of CBDA)
b. 1/3000 dilution of extract—3 UL (stock material is NT1164—3 UL, which equates to 6 μg/ml of CBDA)
c. 1/10000 dilution of extract—1 UL (stock material is NT1164—1 UL, which equates to 0.1 μg/ml of CBDA).

For the CBD sample, a pure standard (in powder form) was used. CBD 98% isolate was purchased as a reference standard from LGC Standards (London UK) (CAS No. 13956-29-1). The CBD standard reference was prepared at concentration of 1 mg/ml (in acetonitrile). CBD dilutions were made in acetonitrile as follows: 2 μg/ml; 6 μg/ml; and 0.1 μg/ml.

The final concentration of NT1164 (CBDA equivalent) and CBD that was used in these studies was 2 μg/ml.

### C.2.2 Microglial BV2 Culture

The immortalized microglia cell line, BV2, was purchased from the American Tissue Culture Collection. BV2 were cultured in RPMI media containing gentamycin and supplemented with 10% FBS for expansion and 5% fetal bovine serum (FBS) when plated for experiments. All cells were from between passage numbers 39 and 45. Cells were plated at 45,000 cells/mm² and treated 24 hours after plating with phosphate buffered saline (PBS, as a control) or interleurkin-1B+interferon-y (IL-1B+IFNγ, to induce inflammation). To test the effects of NT1164 to after the inflammatory response, NT1164 was applied one hour before inflammation (pre-treat) or one hour after (post-treat). NT1164 was applied at 10 μL, 3 uL or 1 uL from isolated obtained using the original extraction protocol: range determined from mass spec data=1.0-0.1 ug CBDA.

### C.2.3 Multiplex Cytokine/Chemokine Assay

Microglia media were harvested following treatment initiation was centrifuged briefly to remove particulates (300 g for 10 min). Cytokine and chemokine levels in the microglial media were measured using a Bio-Plex 200 with a 96-well magnetic plate assay according to the manufacturer's instructions (Bio-Rad). Cytokines and chemokines measured included IL-1α, IL-1β, IL-2, IL-6, IL-10, IL-12 (p70), IL-13, IL-17, G-CSF, GM-CSF, IFNγ, TNFα, CXCL1 (KC), CCL2 (MCP-1), and CCL5 (RANTES). All samples were run in duplicate and data were analyzed with the Bio-Plex Manager software.

### C.2.4 Immunohistochemistry (Protein Level) Assay

Cells were fixed for 10 min with 4% paraformaldehyde (PFA) in PBS. After 3×5 minutes washing with PBS, cells were incubated with primary antibodies (anti-COX2, anti-ARG1) 1:1000 overnight at 4° C., and after 3×5 minutes washing in PBS, cells were then incubated in appropriate fluorescent secondary antibody 1:250 (Invitrogen) for 2 hours at room temperature. After a final wash, as previous, cells nuclei were stained with DAPI in the mounting media. Photomicrographs were taken of the cells in three fields of view per well from duplicate wells and analysed using Fiji for area coverage of each marker.

### C.2.5 Cell Viability (Mitochondrial Activity) Assay

Microglial viability was quantified using MTT [3-(4,5-dimethylthiazol-2-yl-)-2,5-diphenyl-2H-tetrazolium bromide; Sigma]. In this assay MTT, a tetrazolium dye, is bioreduced by the mitochondria into a formazan product that is insoluble in tissue culture medium. In brief, MTT was added to a final concentration of 250 μg/ml to cells at various time points following treatment with PBS, LPS or IL-4 with or without test product. After 30 min, formazan was dissolved in DMSO and the absorbance was measured at 490 nm using a spectrophotometer (Glomax Multi+; Promega, UK).

### C.2.6 Statistics

Data for replicates within experiments were averaged and then data from at least three independent experiments was analysed using Graph Pad Prism or students-t-test.

### C.3 Results

### C.3.1 INOS Expression

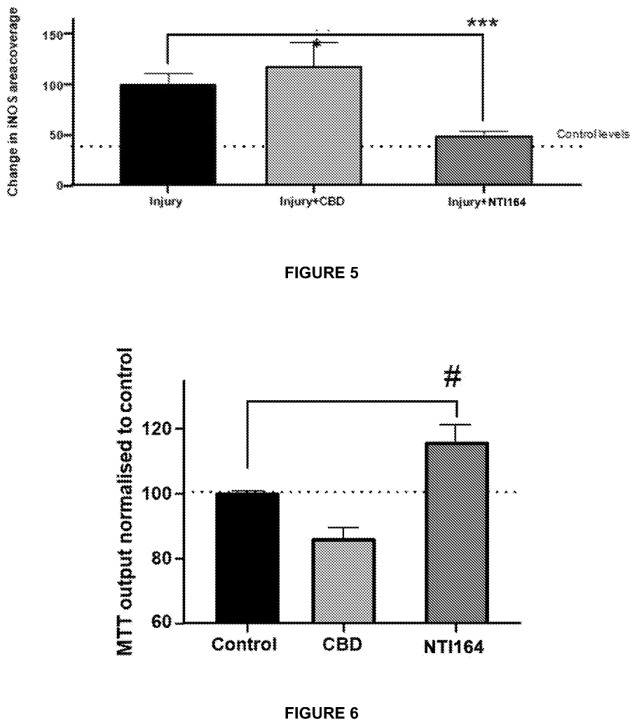

NT1164 normalised inflammation induced iNOS expression. iNOS expression is increased by inflammation and in inflammatory activated microglial cells, NT1164 normalized expression towards control levels, and therefore reduced the inflammatory process triggered by iNOS. Inducible nitric oxide synthase (iNOS) is one of three key enzymes generating nitric oxide (NO) from the amino acid L-arginine. Inducible nitric oxide synthase (iNOS) plays a critical role in the regulation of multiple sclerosis (MS) and experimental autoimmune encephalomyelitis (EAE). Previous studies have shown that iNOS plays pathogenic as well as regulatory roles in MS and EAE and many other neuro-inflammatory disorders. FIG. 5 demonstrates that NT1164 normalised inflammation induced iNOS expression.

### C.3.2 Neuronal Viability

NT1164 increased the number of viable neurons under basal conditions (short term exposure). Neuronal viability was quantified using MTT [3-(4,5-dimethylthiazol-2-yl-)-2,5-diphenyl-2H-tetrazolium bromide; Sigma]. NT1164 treated celled were able to increase the number of “healthy” cells under basal conditions following short term glutamate exposure. Cellular excitotoxicity was achieved via glutamate activation (3 mM). NT1164 was able to stimulate cell growth after short term glutamate induced “insult”.

The Cell Viability (Mitochondrial Activity) Assay (or MTT assay) was used to determine the cellular viability or metabolic activity in microcapsules within the cells. It is based on the ability of metabolically active cells to transform a water-soluble dye[3-(4,5-dimethylthiazol-2-yl)-2,5-diphenyltetrazolium bromide] into an insoluble formazan. Cell viability is a measure of the proportion of live, healthy cells within a population and cell viability assays are used to determine the overall health of cells. FIG. 6 presents the neuronal viability quantified using MTT [3-(4,5-dimethylthiazol-2-yl-)-2,5-diphenyl-2H-tetrazolium bromide].

### C.3.3 Maturation of Immature Neurons

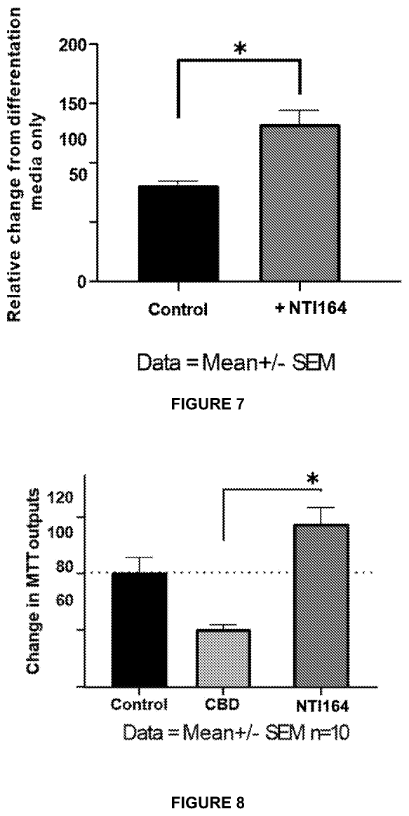

NT1164 stimulated the maturation of immature neurons into healthy cells even without the presence of any Glutamate induced insult. This study demonstrates that NT1164 can stimulate “healthy maturation” of immature neurons. This is process that may be vital after trauma, or damage. NT1164 is able to provide healthy neuronal ell development which is a vital process in recovery from neuro-inflammation, neuronal damage. FIG. 7 demonstrates that NT1164 (NTI strain) stimulates the maturation of immature neurons into healthy cells even without the presence of any glutamate induced insult.

### C.3.4 Cell Death

NT1164 does not increase cell death in an excitotoxic cell injury paradigm.

FIG. 7 demonstrates that CBD is toxic in this paradigm while NT1164 (is non-toxic and has positive effects on cell number and cell viability.

### C.3.5 ARG1 Expression

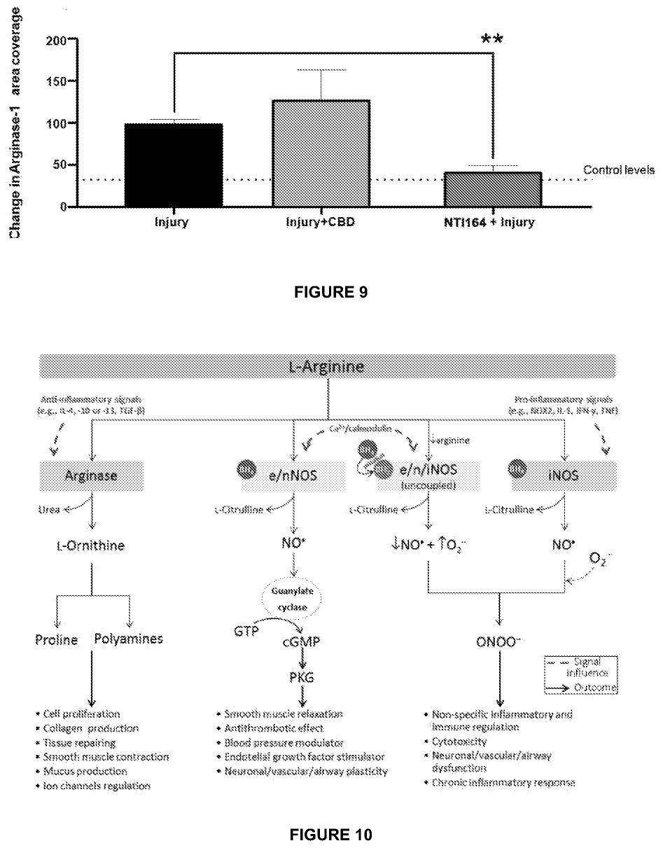

NT1164 normalises inflammation-induced (injured cells) Arg 1 expression. Macrophage specific upregulation of Arginase-1 is commonly believed to promote inflammation. Arginase 1 expression is increased by inflammation yet in inflammatory activated cells, NTI164 normalizes expression towards control levels. FIG. 9 shows the microglial responses under inflammatory conditions assessing Arginase 1 expressions.

FIG. 10 outlines the Arginine metabolism and the effects it has on the overall balance of anti-inflammatory and pro-inflammatory signals.

### D Example 4—Preclinical Studies Relating to Biomarkers Involved in Neuroinflammation

### D.1 Study Aim

To determine the effect of NT1164 on the levels of COX-2, IL2 and TNF-alpha in human derived microglial cells.

Many neurological disorders occur due to inflammation which is induced by dysregulation of immune responses.

For example, multiple sclerosis (MS) is a progressive inflammatory disease characterised by the loss of myelin sheath within the central nervous system. Typical symptoms include fatigue, walking difficulties and impaired speech and vision. Cyclooxygenase-2 (COX-2) is considered the main enzyme responsible for causing inflammation, the common mechanism of disease involved in MS. COX-2 is a powerful clinical biomarker in the assessment of disease progression and overall therapeutic management.

IL2 plays an important role in immune regulation and an important role in MS progression. IL-12 is a cytokine that plays a key role in the pathogenesis of Multiple Sclerosis. Blocking this cytokine via a neutralizing antibody causes dramatic improvements in animal models of the disease, and multiple human trials.

TNF-alpha plays an important role plays an important role in dysregulation of acute inflammation involved in MS onset.

### D.2 Materials and Methods

### D.2.1 COX-2

Immunohistochemistry (Protein Level) Assay: Cells were fixed for 10 min with 4% paraformaldehyde (PFA) in PBS. After 3×5 minutes washing with PBS, cells were incubated with primary antibodies (anti-COX2) 1:1000 overnight at 4° C., and after 3×5 minutes washing in PBS, cells were then incubated in appropriate fluorescent secondary antibody 1:250 (Invitrogen) for 2 hours at room temperature. After a final wash, as previous, cells nuclei were stained with DAPI in the mounting media. Photomicrographs were taken of the cells in three fields of view per well from duplicate wells and analysed using Fiji for area coverage of each marker.

### D.2.2 IL-2 and TNF-Alpha

Multiplex Cytokine/Chemokine Assay: Microglia media harvested following treatment initiation was centrifuged briefly to remove particulates (300 g for 10 min). Cytokine and chemokine levels in the microglial media were measured using a Bio-Plex 200 with a 96-well magnetic plate assay according to the manufacturer's instructions (Bio-Rad). Cytokines and chemokines measured included IL-1α, IL-1β, IL-2, IL-6, IL-10, IL-12 (p70), IL-13, G-CSF, GM-CSF, IFNγ, TNFα, CXCL1 (KC), CCL2 (MCP-1), and CCL5 (RANTES). All samples were run in duplicate and data were analyzed with the Bio-Plex Manager software.

### D.2.3 Sample Preparation and Dilutions

500 mg of dried plant material of NT1164 is suspended in 20 ml of absolute ethanol (using 50 ml blue top falcon tubes appropriate for centrifugation) and stirred/shaken vigorously for 60 seconds. The tubes are then placed in a sonication bath for 10 mins at 35-40 C. Upon completion of the sonication samples are then placed in a tray shaker (200 rpm) for 30 minutes at room temperature. Once completed the samples are then centrifuged at 4400 rpm for 5 minutes. The supernatant is collected for testing and development.

Units used to describe treatments for test products and concentrations for NT1164.
a. 1/1000 dilution of extract—10 UL (stock material is NT1164—10 UL, which equates to 2 μg/ml of CBDA)
b. 1/3000 dilution of extract—3 UL (stock material is NT1164—3 UL, which equates to 6 μg/ml of CBDA)
c. 1/10000 dilution of extract—1 UL (stock material is NTI164—1 UL, which equates to 0.1 μg/ml of CBDA)

For the CBD sample, a pure standard (in powder form) was used. CBD 98% isolate was purchased as a reference standard from LGC Standards (London UK) (CAS No. 13956-29-1). The CBD standard reference was prepared at concentration of 1 mg/ml (in acetonitrile). CBD dilutions were made in acetonitrile as follows: 2 μg/ml; 6 μg/ml; and 0.1 μg/ml.

The final concentration of NT1164 (CBDA equivalent) and CBD that was used in these studies was 2 μg/ml.

### D.3 Results

### D.3.1 COX-2

The preclinical studies conducted in cells using immunohistochemistry analysis demonstrated that NT1164 can suppress and inhibit the expression of COX-2 in human derived microglial cells. When compared to CBD alone, NT1164 was up to three times more powerful in suppressing COX-2 both pre and post inflammatory insult. Refer to Table 7 below.

TABLE 7
Outlines the COX-2 suppression in cells when treated with NTI164 versus CBD alone.

| | | | | | |
|---|---|---|---|---|---|
| | | | | Positive control | Positive control |
| | | | | vs NTI164 | vs CBD alone |
| N | Control Avg | NTI164 | CBD Avg | treatment | treatment |

Pre-Inflammatory Exposure (exposure
1 hour prior to inflammatory insult)

| | | | | | |
|---|---|---|---|---|---|
| 9 | 94.47 +/− 5.90 (SEM) | 53.67 +/− 6.41 (SEM) | 84.82 +/− 7.65 (SEM) | P = 0.0003 | P = 0.3237 |

Pre-Inflammatory Exposure (exposure
1 hour after inflammatory insult)

| | | | | | |
|---|---|---|---|---|---|
| 9 | 104.26 +/− 11.08 (SEM) | 21.10 +/− 6.82 (SEM) | 76.32 +/− 7.95 (SEM) | P < 0.0001 | P = 0.0566 |

NTI164 is more potent than CBD alone in suppressing COX-2 expression in human microglial cells.
DAPI cell viability stain: No cell death was detected and assessed as per the DAPI cell staining method.
Cells were viable throughout these in vitro studies.
Positive control/Inflammatory activation: Interleukin and Interferon gamma

### D.3.2 IL2 and TNF-Alpha

NT1164 is statistically more potent in suppressing the key biomarkers: IL-12 and TNF-alpha when compared CBD alone and CBD|THC (1:1) mixture.

These results demonstrate that: NT1164 IL-12 P=0.0011 versus CBD alone was highly significant; NT1164 TNF-alpha P=0.0575 versus CBD alone was a positive trend; NT1164 IL-12 P=0.0069 versus CBD\THC combination Highly significant; NT1164 TNF-alpha P=0.0446 versus CBD\THC combination was significant.

TABLE 8
outlines the significance between NTI164 versus CBD alone and CBD \
THC combinations (1:1 concentration ratio) in suppressing TNF-alpha and IL-12.

| | | | Statistical Significance |
|---|---|---|---|
| | | Results Expressed as: | Reduction |
| | | Average +/− Standard | Compared to Control |
| Treatment | Cytokine | Deviation (SD) | Alone |
| Control: Interleukin and | IL-12 | 99.91 +/− 12.88 | |
| Interferon Activation | TNF -alpha | | |
| NTI164 (2 μg/ml) | IL-12 | 56.30 +/− 18.24 | *P = 0.0001 |
| | TNF -alpha | 58.28 +/− 15.08 | *P < 0.0001 |
| CBD alone (2 μg/ml) | IL-12 | 84.40 +/− 6.54 | P = 0.008 |
| | TNF -alpha | 71.13 +/− 12.81 | P = 0.0005 |
| CBD \| THC (1:1) | IL-12 | 80.77 +/− 12.23 | P = 0.008 |
| (2 μg/ml) | TNF -alpha | 74.49 +/− 14.30 | P = 0.01 |

### D.4 Discussion

### D.4.1 COX-2. IL2 and TNF-Alpha

These results, showing suppressing COX-2, IL2 and TNF-alpha, reconfirm the potent properties of NT1164 in modulating inflammatory processes in neurological disorders where inflammation induced by immune responses is dysregulated.

### E Example 5—Further Characterisation of NT164

### E.1 Study Aim

The compositional analysis of NT1164 extracted via oil, using UPLC/MS methods (as outlined in the above examples).

### E.2 Materials and Methods

The ACQUITY UPLC H-Class System combined with the CORTECS UPLC Shield RP18 particle chemistry was used to provide a UPLC isocratic separation of main cannabinoids in a 10.5-minute cycle time. Samples of NT1164 were assayed on a weekly basis and CBDA was used as a main marker as a stability indicator. Results presented in Table 6 demonstrate that NT1164 is stable at room temperature within an inert oil media over 6 weeks. There is no decarboxylation or product degradation observed over this time frame

Equipment: The following equipment was used: 10 mL glass scintillation bottles with lids; Cobram's Estate olive oil; plant grinder (similar to a coffee or food grade grinder) pore size up to 50 μM-80 μM; Whatman paper, grade 1; pipettes; weight scale (transfer boats and spoons); Eppendorf tubes; 50 mL falcon tubes; bench top centrifuge (Eppendorf Centrifuge 5702); Oz Design Brand 6 Litre Fruit, Wine and Cider Press.

Extraction: Pressing and Centrifugation: All work is undertaken at standard lab temperatures (18-22° C.). The buds of NT1164 were stripped off hard stalks and the stalks discarded. The grinder was cleaned with 70% EtOH and the grinding compartment was filled with dried plant material. The material was ground on the finest of the three setting for 10 seconds (1-2 mm particle size). The grounds were then mixed with 100 ml of olive oil in an autoclaved Schott bottle at a ratio plant/oil of 333 mg/ml. It was then placed on a stirrer at room temperature for 1 hour, stirred with magnetic flea (50 rpm). The oil plus plant mixture is then put into the Oz Design Brand 6 Litre Fruit, Wine and Cider Press to reclaim the oil component from the plant (the mash). The reclaimed oil was then placed into 50 mL falcon tubes and spun at 300 g for 15 minutes at room temperature (Isolation 1). The oil was then removed into a clean Schott bottle and keeping track of the volume reclaimed. The recovery of the oil for Isolation 1 is approximately 40%. The mash is discarded following each isolation. To the reclaimed oil, we added a further 333 mg/mL ground plant/oil (a further 100 ml) material and repeated the 1 hour mix, and reclaimed and re-used oil, until a total of 999 μg/mL (3×100 ml) of plant/oil mixture passed through (Isolation 2). The recovery of the oil for Isolation 2 is approximately 50%. For the final time, we placed into falcon tubes and spin as discussed above (Isolation 3). The recovery of the oil at for Isolation 3 is approximately 50%. We then collected the oil only and placed the oil into Eppendorf tubes for processing. This triplicate extraction method resulted in a total volume of 50 ml of final product at a concentration of 48 mg of CBDA to 1 ml of olive oil determined using UPLC potency testing using the methods described below.

### E.3 Results

The results of the UPLC analysis are presented below in Table 9.

TABLE 9
presents the NTI164 composition extracted using
the ethanol extraction and the components quantified
using the UPLC methods herein described

| | | Oil | |
|---|---|---|---|
| | | Extract | Oil |
| | | 18 Nov. 2021 | Extract |
| | | (NTI164) | 7 Dec. 2021 |
| CANNABINOIDS | Cas No's | % w/w | % w/w |
| Cannabidiol (CBD) | 13956-29-1 | 5.80 | 5.34 |
| Cannabidiol acid (CBD-A) | 1244-58-2 | 62.78 | 60.29 |
| Tetrahydrocannabinol | 1972-08--03 | 0.70 | 0.65 |
| (D9-THC) | | | |
| (Tetrahydrocannabinolic | 23978-85-0 | 2.01 | 1.99 |
| acid) (D9- THC-A) | | | |
| Cannabidivarin (CBDV) | 24274-48-4 | 0.49 | 0.48 |
| Cannabigerol Acid(CBG-A) | 25555-57-1 | 1.26 | 1.14 |
| Cannabigerol (CBG) | 25654-31-3 | 0.44 | 0.39 |
| Tetrahydrocannabivarin | 28172-17-0 | 0.26 | 0.26 |
| (THCV) | | | |
| Cannabinol (CBN) | 521-35-7 | 1.98 | 2.28 |
| DELTA-8-THC (D8-THC) | 5957-75-5 | <0.1 | <0.1 |
| Cannabichrome (CBC) | 20675-51-8 | 0.81 | 0.85 |
| Total THC = D9-THC + | % amount | 2.45 | 2.40 |
| (D9-THC-A*0.877) | | | |
| Total CBD = CBD + | % amount | 60.85 | 58.22 |
| (CBD-A*0.877) | | | |

By expanding the pore size in the grinder system from 50 μM to 80 μM, we were able to extract cannabinol (CBN) 1-3% in the final oil extract product.

### F Example 6—NTI164 for Treatment of ASD Level II/III—4 Weeks

### F.1 ASD Study Design and Methods

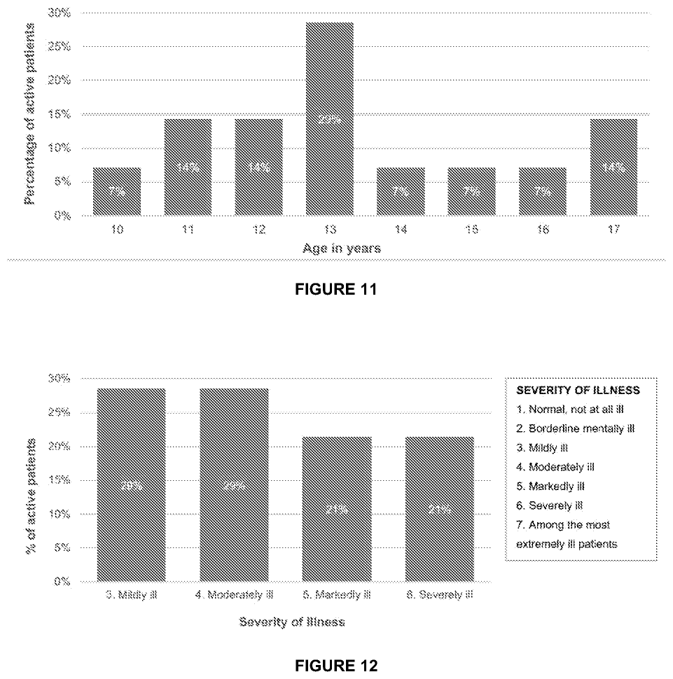

Of the 18 patients who were enrolled into the study, patients who received NT1164 made up 94% (n=17). Active patients made up 78% (n=14), patients who discontinued after receiving their first dose of NT1164 made up 16% (n=3) and patients who discontinued prior to receiving NT1164 but after enrolment made up 6% (n=1). The mean age of active patients was 13.4 years of age with the youngest patient being 10 years and the oldest being 17 years of age (FIG. 11).

All active patients were diagnosed with ASD Level II/III and were assessed at baseline as being either ‘Mildly ill’, ‘Moderately ill’, ‘Markedly ill’ or ‘Severely ill’ on the CGI Severity scale (FIG. 12).

Patients commenced treatment of NT1164 at 5 mg/kg/day and was increased weekly by 5 mg/kg/day for a period of 4 weeks until 20 mg/kg/day or the maximum tolerated dose was achieved. The daily dose was calculated by multiplying the dosage by the patient's weight and then dividing by the concentration of CBDA in the oil (53 mg/mL). This returned a total daily volume in mL (Table 10) which was split into twice daily (BD) AM and PM doses.

TABLE 10
Calculation of daily dose for each patient.

| | | Daily Dose in mL | | | |
|---|---|---|---|---|---|
| Patient ID | Weight | 5 mg/kg | 10 mg/kg | 15 mg/kg | 20 mg/kg |
| 102 | 56.9 | 5.4 | 10.7 | 16.1 | 21.4 |
| 103 | 57.8 | 5.4 | 10.8 | 16.3 | 21.7 |
| 104 | 57.4 | 5.3 | 10.6 | 15.8 | 21.7 |
| 106 | 41.0 | 3.8 | 7.7 | 11.5 | 15.4 |
| 110 | 91.7 | 8.7 | 17.3 | 26.0 | 34.0 |
| 111 | 41.1 | 3.9 | 7.9 | 11.8 | 15.7 |
| 112 | 101.1 | 9.5 | 19.1 | 28.6 | 38.2 |
| 113 | 44.7 | 4.2 | 8.4 | 12.7 | 16.9 |
| 114 | 89.8 | 8.5 | 16.9 | 25.4 | 33.9 |
| 115 | 40.5 | 3.7 | 7.5 | 11.2 | 14.9 |
| 116 | 54.5 | 5.1 | 10.2 | 15.2 | 20.3 |
| 117 | 65.5 | 6.3 | 12.6 | 18.8 | 24.7 |
| 118 | 52.4 | 4.9 | 9.9 | 14.8 | 19.8 |
| 119 | 68.9 | 6.5 | 13.0 | 19.5 | 26.0 |

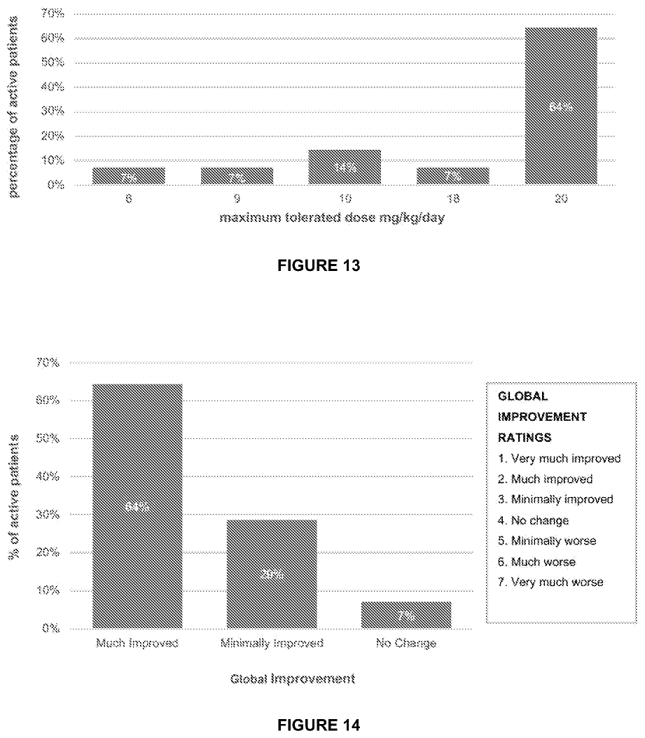

The average maximum daily dose for active patients was 16.7 mg/kg/day with 64% of patients tolerating the maximum dose of 20 mg/kg/day and 36% of patients tolerating a maximum daily dose ranging between 6 mg/kg/day to 19 mg/kg/day (FIG. 13).

### F.2 Study Results

The Clinical Global Impression-Severity (CGI-S) scale was used to assess:
Global Improvement: rates the total improvement whether or not, in the clinician's judgement, is due entirely to drug treatment;
Severity of Illness: a comparison of baseline and post-baseline (28-days NT1164 treatment); and
Efficacy Index: rated based on drug effect only. This is a calculated score based on the degrees of therapeutic effect and side effects.

### Global Improvement

93% of active patients showed improvement after 28 days of daily treatment with NT1164. 64% of these patients had a global improvement of ‘Much improved’, 29% had a global improvement of ‘Minimally improved’ and only one patient (7%) had ‘No change’ (FIG. 14). The Wilcoxon Signed-Rank Test and the Paired t-test were used to assess the statistical significance:

Paired t-test: the mean difference of CGI-S between 28 days of treatment and baseline was −0.714, 95% confidence interval=−1.332, −0.097, p value=0.027. The Wilcoxon Signed-Rank Test statistic was: −15, the corresponding p-value was 0.047.

### Severity of Illness

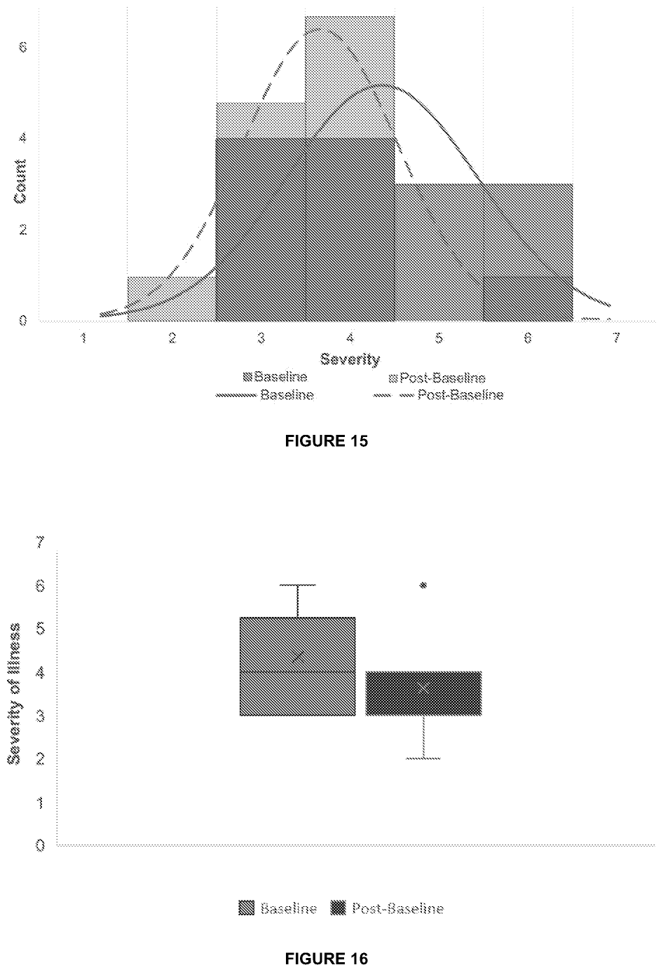

The average rating for the severity of illness at baseline was 4.4 (FIG. 12). This reduced to an average rating of 3.6 after 28 days of NT1164 treatment (FIGS. 15, 16).

### Therapeutic Effect

After 28-days of daily treatment with NT1164, 14% of active patients demonstrated the second highest possible efficacy index of 2: Marked therapeutic effect with side effects that do not significantly interfere with patient's functioning.

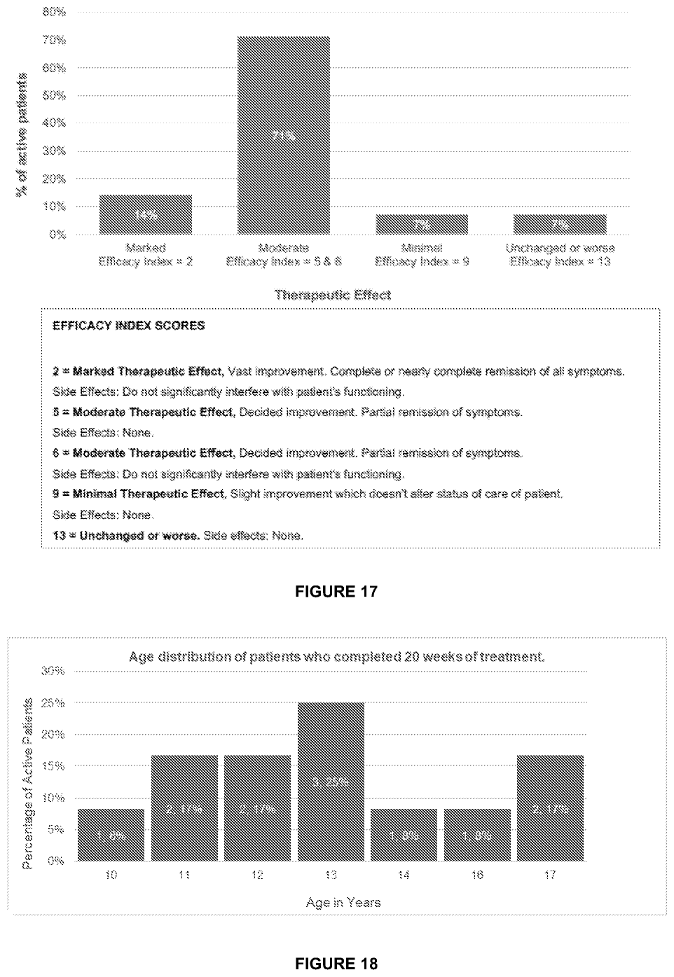

72% of active patients had an efficacy index of either 5 or 6: Moderate therapeutic effect with half of these patients having no side effects and the other half having side effects that do not significantly interfere with patient's functioning, 7% had an efficacy index of 9: Minimal therapeutic effect with no side effects and only one patient, 7%, had an efficacy index based on seeing no change in condition, 13: Unchanged or worse with no side effects (FIG. 17).

### G Example 7—NT1164 for Treatment of ASD Level II/III—20 Weeks

### G.1 ASD Study Design and Methods

Example 6 above presents the results of the treatment at the 4 weeks (28 days) time point (n=14 active). This Example 7 presents the results of the treatment at the 20 week time point (n=12 active). As discussed in Example 6 above, patients commenced treatment of NTI164 at 5 mg/kg/day which was increased weekly by 5 mg/kg/day for a period of 4 weeks until 20 mg/kg/day or the maximum tolerated dose was achieved and (in this study) continued their maximum tolerated dose for 16 weeks (providing a total daily dosing period of 20 weeks).

The overall purpose of this study was to assess the continued safety and efficacy of NT1164 administered daily over a 20-week period. The secondary objective was to assess the efficacy of NT1164 in the treatment of symptoms associated with autism spectrum disorder. Efficacy was measured with various physician-led and parent-led standard questionnaires used in the art.

Patients (n=12)

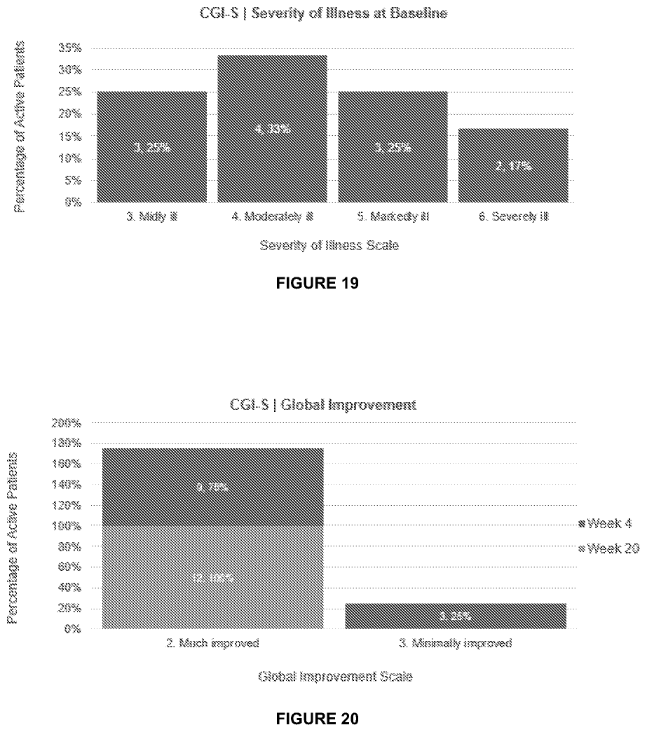

The mean age of active patients at week 20 was 13.3 years of age with the youngest patient being 10 years and the oldest being 17 years of age (Error! Reference source not found.). All active patients were diagnosed with ASD Level II/III and were assessed at baseline as being either ‘Mildly ill’, ‘Moderately ill’, ‘Markedly ill’ or ‘Severely ill’ on the CGI Severity scale (FIG. 19).

### Dose

Based on paediatric trials undertaken around the world, the selected maximum dose for this study was 20 mg/kg/day.

To reduce the risk of side-effects, the study drug was up-titrated over the course of four weeks commencing at 5 mg/kg/day and increasing weekly by 5 mg until the maximum tolerated dose or 20 mg/kg/day was achieved. The maximum tolerated dose was then administered over the course of 20 weeks. The daily volume was administered over two doses, AM and PM.

The formula used to calculate each patient's dose was: Weight×Dose/NT1164 Concentration=Daily Dose/2=Twice-Daily Dose. During the first week of treatment, each patient received 5 mg/kg/day of NT1164. During the second week of treatment, each patient received 10 mg/kg/day of NT1164. During the third week of treatment, each patient received 15 mg/kg/day of NT1164. During the fourth week of treatment, each patient received 20 mg/kg/day of NT1164. During weeks 5-20 of treatment, each patient received their maximum tolerated dose or 20 mg/kg/day of NT1164.

NT1164 was prepared in oil for oral administration. The total concentration of the oil was 53 mg/ml.

At the end of week 20, participants had the option of either ending their participation and down-titrating 5 mg/kg/week until they ceased the study drug or continuing their maximum tolerated dose up to week 52.

### Primary Endpoints

Safety was monitored and measured using standard steps in the art. Safety was monitored and measured using full blood examinations, liver and kidney function tests and vital signs in addition to parent/carer and physician questionnaires completed at baseline and every four weeks up until week 20.

### Secondary Endpoints

Efficacy was monitored and measured using standard steps in the art.

Clinical Global Impression Scale-Severity of Illness (CGI-S). Reflects clinician's impression of severity of illness on a 7-point scale ranging from 1=not at all to 7=among the most extremely ill. [Time Frame: Baseline, Week 4, Week 8, Week 12, Week 20].

Vineland Adaptive Behaviour Scales, Third Edition (Vineland-3). Used to measure adaptive functioning across three core domains (Communication, Daily Living Skills, and Socialization), and two optional domains (Motor Skills and Maladaptive Behaviour); items are rated on a 3-point scale (0=never; 1=sometimes; 2=usually or often). The core domains sum to a total Adaptive Behaviour Composite. [Time Frame: Baseline, Week 20].

Social Responsiveness Scale, 2nd Edition—School—Age Form (SRS-2). Five domains are assessed including: Social Awareness, Social Cognition, Social Communication, Social Motivation, and Restricted Interests and Repetitive Behaviour. Items are scored on a 4-point scale (ranging from 1=not true to 4=almost always true). [Time Frame: Baseline, Week 20].

Clinical Global Impression Scale-Improvement-Caregiver (CGI-I-Ca). This is a 7-point scale measuring symptom change from baseline.

Provided as baseline and post-baseline Caregiver and Clinician questionnaires. [Time Frame: Baseline, Week 4, Week 8, Week 12, Week 20].

Clinical Global Impression Scale-Improvement-Clinician (CGI-I-Cl). This is a 7-point scale measuring symptom change from baseline.

Provided as baseline and post-baseline Caregiver and Clinician questionnaires. [Time Frame: Baseline, Week 4, Week 8, Week 12, Week 20].

Clinical Global Impression Scale-Change in Target Behaviour (CGI-C). Reflects clinician's impression of change of behaviour on a 7-point scale ranging from 1=not at all to 7=very severe problem. Provided as Baseline and Post-Baseline questionnaires. [Time Frame: Baseline, Week 4, Week 8, Week 12, Week 20].

Clinical Global Impression Scale-Change in Attention (CGI-CA). Reflects clinician's impression of change in attention on a 7-point scale ranging from 1=not at all to 7=very severe problem. Provided as Baseline and Post-Baseline questionnaires. [Time Frame: Baseline, Week 4, Week 8, Week 12, Week 20].

Anxiety Scale for Children-Autism Spectrum Disorder-Parent Version (ASC-ASD-P). Parent/Caregiver form developed to detect symptoms of anxiety in youth with ASD. Composed of four subscales (Performance Anxiety, Uncertainty, Anxious Arousal, and Separation Anxiety), items are rated on a 4-point scale (0=never and 3=always). Subscales sum to equal a total score. [Time Frame: Baseline, Week 4, Week 8, Week 12, Week 20].

Anxiety Scale for Children-Autism Spectrum Disorder-Child Version (ASC-ASD-C). Child form developed to detect symptoms of anxiety in youth with ASD. Composed of four subscales (Performance Anxiety, Uncertainty, Anxious Arousal, and Separation Anxiety), items are rated on a 4-point scale (0=never and 3=always). Subscales sum to equal a total score. [Time Frame: Baseline, Week 4, Week 8, Week 12, Week 20].

Sleep Disturbance Scale for Children (SDSC). Six subscales including Disorders of Initiating and Maintaining Sleep, Sleep Breathing Disorders, Disorders of Arousal, Sleep Wake Transition Disorders, Disorders of Excessive Somnolence, and Sleep Hyperhydrosis. Items are rated on 5-point scale where 1=never and 5=always (daily). Subscale scores sum to equal a total score [Time Frame: Baseline, Week 4, Week 8, Week 12, Week 20].

### G.2 Study Results

### Safety Results

The safety data concludes that NT1164 at 5, 10, 15 and 20 mg/kg administered in two doses daily, is safe and well-tolerated in this study population. This conclusion is further supported by laboratory values. No changes were observed to patient's full blood examination, liver function or kidney function tests. Nor were there any changes observed to patient's vital signs.

### Efficacy Results

The Wilcoxon Signed-Rank Test and the Paired t-test were used to assess statistical significance of the analysed data sets.

Paired t-test: the mean difference of CGI-S between 20 weeks of treatment and baseline was −1.08, 95% confidence interval=−1.772, −0.3948, p value=0.005303.

The Wilcoxon Signed-Rank Test statistic was: −15, the corresponding p-value was 0.009654.

100% of patients (n=12) showed ‘much improved’ improvement of symptoms relating to severity of illness after 20 weeks of daily treatment with NT164.

Table 15 below is a summary of results from the Wilcoxon Signed-Rank Tests and paired T-Tests on analysed datasets at week 20.

TABLE 15
Summary of Wilcoxon Signed-Rank Test and Paired
T-Test on Analysed Datasets at 20 weeks

| | | Wilcoxon | |
|---|---|---|---|
| | | Signed- | Paired |
| Sub-Domain | Scale | Rank Test | T-Test |
| Severity of illness | CGI-S | 0.010 | 0.005 |
| Adaptive behaviour composite (Total) | Vineland-3 | 0.003 | 0.001 |
| Communication | Vineland-3 | 0.004 | 0.002 |
| Daily living skills | Vineland-3 | 0.025 | 0.019 |
| Socialisation | Vineland-3 | 0.012 | 0.014 |
| Social responsive scale - Total | SRS-2 | 0.013 | 0.012 |
| T-score | | | |
| Social awareness - T-score | SRS-2 | 0.439 | 0.596 |
| Social cognition - T-score | SRS-2 | 0.036 | 0.028 |
| Social communication - T-score | SRS-2 | 0.018 | 0.019 |
| Social motivation - T-score | SRS-2 | 0.138 | 0.118 |
| Restricted interest and repetitive | SRS-2 | 0.014 | 0.009 |
| behaviour - T-score | | | |
| Social communication and | SRS-2 | 0.021 | 0.029 |
| interaction - T-score | | | |
| Anxiety scale for children - | ASC-ASD-C | 0.012 | 0.025 |
| Child's total | | | |
| Performance anxiety | ASC-ASD-C | 0.474 | 0.364 |
| Anxious arousal | ASC-ASD-C | 0.089 | 0.120 |
| Separation anxiety | ASC-ASD-C | 0.035 | 0.025 |
| Uncertainty | ASC-ASD-C | 0.035 | 0.033 |
| Anxiety scale for children - | ASC-ASD-P | 0.053 | 0.034 |
| Parent's total | | | |
| Performance anxiety | ASC-ASD-P | 0.096 | 0.070 |
| Anxious arousal | ASC-ASD-P | 0.229 | 0.333 |
| Separation anxiety | ASC-ASD-P | 0.033 | 0.025 |
| Uncertainty | ASC-ASD-P | 0.084 | 0.066 |
| Sleep disturbances scale for | SDSC | 0.018 | 0.016 |
| children - Total | | | |
| Disorders of initiating and | SDSC | 0.026 | 0.010 |
| maintaining sleep | | | |
| Sleep breathing disorders | SDSC | 0.042 | 0.047 |
| Disorders of arousal | SDSC | 0.480 | 0.522 |
| Sleep-wake transition disorders | SDSC | 0.072 | 0.094 |
| Disorders of excessive somnolence | SDSC | 0.706 | 1.000 |
| Sleep hyperhydrosis | SDSC | 1.000 | 1.000 |
| Anxiety, depression and mood | ADAMS | 0.009 | 0.001 |
| scale - Total | | | |
| Autism family experience | AFEQ | 0.888 | 0.961 |
| questionnaire - Total | | | |
| Experience of being a parent of | AFEQ | 0.443 | 0.464 |
| a child with autism | | | |
| Family life | AFEQ | 0.234 | 0.247 |
| Child development, understanding | AFEQ | 0.765 | 0.799 |
| and social relationships | | | |
| Child symptoms (feelings and | AFEQ | 0.119 | 0.120 |
| behaviour) | | | |

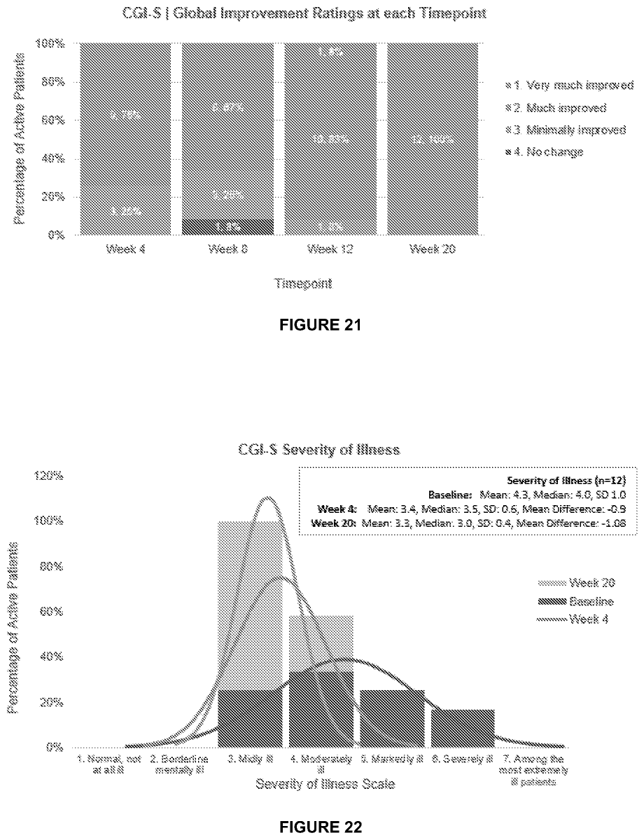

Global Improvement. 100% of active patients (n=12) showed improvement after 20 weeks of daily treatment with NTI164. All patients had a global improvement of ‘2. Much improved’. Three of these patients had previously scored ‘3. Minimally improved’ after 4 weeks of treatment. See FIGS. 20 and 21.

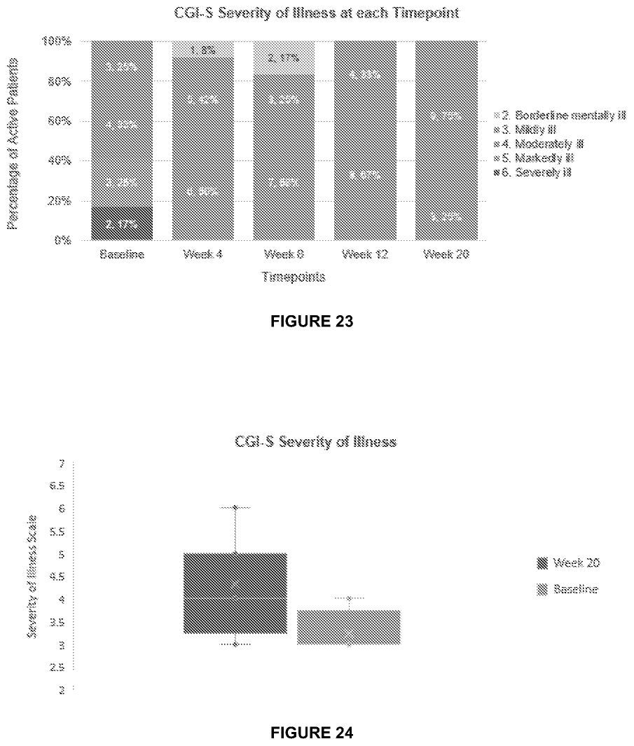

Severity of Illness. The average rating for the severity of illness at baseline was 4.3. This reduced to an average rating of 3.3 after 20 weeks of daily NT1164 treatment. See FIGS. 22 to 24.

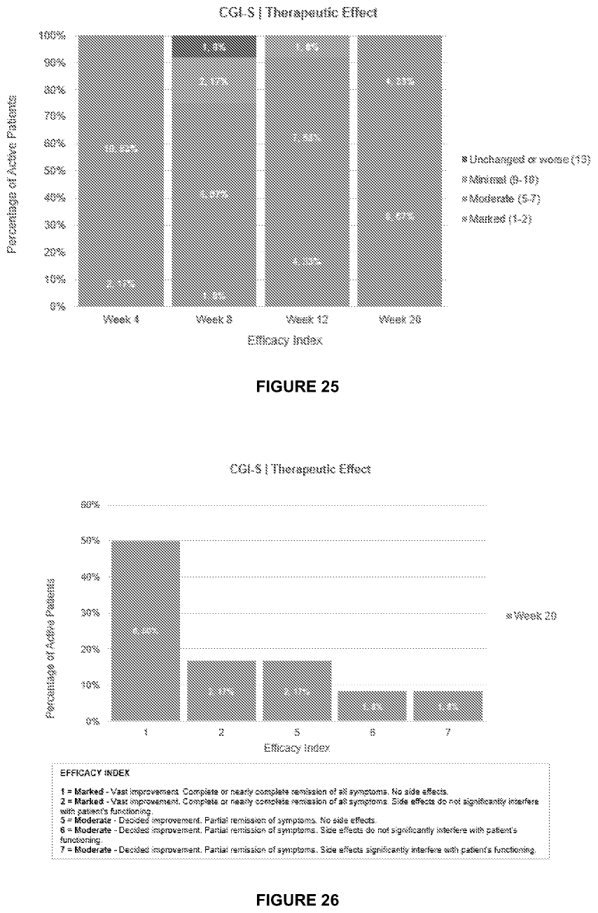

Therapeutic Effect. After 20 weeks of daily NT1164 treatment, 67% of active patients demonstrated the highest possible efficacy indexes of 1 and 2: Marked therapeutic effect—Vast improvement. Complete or nearly complete remission of all symptoms. 33% of patients had an efficacy index of either 5, 6 or 7: Moderate therapeutic effect—Decided improvement. Partial remission of symptoms. See FIGS. 25 and 26.

### CONCLUSION

NT1164 was shown to be safe and well tolerated up to doses of 20/mg/kg/day. NT1164 has shown statistically significant efficacy in improving the symptoms associated with autism spectrum disorder after 20 weeks of daily therapy.

1. A composition comprising the following cannabinoids:
w/w %
CBDA 40-60%;
CBD 1-5%;
CBG 1-10%;
CBDP 1-5%;
CBDB 1-5%;
CBGA 1-10%;
CBN 1-3%
and
THC<1%.

2. The composition of claim 1, wherein the cannabinoids are present in amounts selected from the group consisting of:
Composition 1 comprising
w/w %
CBDA 50%;
CBD 2%;
CBG 5%;
CBDP 2%;
CBDB 2%;
CBGA 5%;
CBN 3% and
THC<0.3%;
and
Composition 2 comprising
w/w %
CBDA 45%;
CBD 1%;
CBG 4%;
CBDP 1%;
CBDB 2%;
CBGA 4%;
CBN 2% and
THC<0.2%.

3. The composition of claim 1, further comprising an oil selected from the group consisting of: a synthetic oil; plant based oil; mineral oil; canola oil; and olive oil.

4. The composition of claim 1, wherein the composition comprises less than 5% w/w terpenes.

5. The composition of claim 1, wherein the composition comprises less than 2% w/w organic plant material.

6. The composition of claim 1, wherein the composition comprises less than 2% w/w of plant phenols.

7. The composition of claim 1, wherein the cannabinoid component of the composition is selected from the group consisting of: between 1 and 500 mg/ml; between 10 and 100 mg/ml; be at a concentration of 50 mg/ml.

8. The composition of claim 1, wherein the CBDA component of the composition is selected from the group consisting of: between 1 and 500 mg/ml; between 10 and 100 mg/ml; be at a concentration of 50 mg/ml.

9. (canceled)

10. A pharmaceutical composition comprising the composition of claim 1 and a pharmaceutically acceptable carrier.

11. A dosage form comprising the composition of claim 1.

12. The dosage form of claim 11, wherein the CBDA component of the composition is selected from the group consisting of: between 1 mg and 1000 mg; between 1 mg and 500 mg; between 1 and 100 mg; less than 400 mg; less than 300 mg; less than 200 mg and less than 100 mg.

13. The dosage form of any one of claim 11, wherein the CBDA component of the composition is selected from the group consisting of: 600 mg; 400 mg; 300 mg; 200 mg; 100 mg; 50 mg; 10 mg; 5 mg; 2 mg; 1 mg.

14. A method of treating a disorder, said method comprising administering to a patient in need thereof a therapeutically effective amount of the dosage form of claim 11.

15. The method of claim 14, wherein the disorder is a neurological disorder.

16. The method of claim 15, wherein the neurological disorder is selected from the group consisting of: Alzheimer's disease (AD); Parkinson's disease (PD); multiple sclerosis; amyotrophic lateral sclerosis; cerebral ischemia; traumatic brain injury; rheumatoid arthritis; chronic migraine; epilepsy; autism spectrum disorder (ASD); attention deficit hyperactivity disorder (ADHD); cerebral palsy and relevant subtypes; neuropathic pain; and depression.

17. (canceled)

18. A process of extracting the composition of claim 1 from cannabis plant material, said process comprising the steps of:
(1) Grinding the cannabis plant material to a sufficient grind size;
(2) Contacting the grind produced by step a) with oil;
(3) Mixing the grind and oil for a sufficient time period to form a mixture;
(4) Pressing the mixture to reclaim the oil;
(5) Centrifuging the oil to further refine the oil; and
(6) Collecting the oil extract in a suitable container.

19. A process of extracting the composition of claim 1 from cannabis plant material, said process comprising the steps of:
(1) Grinding the cannabis plant material to a sufficient grind size;
(2) Contacting the grind produced by step a) with an alcohol;
(3) Mixing the grind and alcohol for a sufficient time period to form a mixture;
(4) Sonicating the mixture;
(5) Centrifuging the mixture; and
(6) Collecting the alcohol extract in a suitable container.

20. A product produced from the process of claim 18.

21. A kit comprising the dosage form of claim 11 and instructions for use.

22. (canceled)

23. A product produced from the process of claim 19.
